(19) United States
(12) Patent Application Publication
(10) Pub. No.: US 2016/0194642 A1
(43) Pub. Date: Jul. 7, 2016

(54) SPHERICAL NUCLEIC ACID-BASED CONSTRUCTS AS IMMUNOREGULATORY AGENTS

(71) Applicant: EXICURE, INC., Skokie, IL (US)

(72) Inventors: Sergei Gryaznov, San Mateo, CA (US); Christopher C. Mader, Cambridge, MA (US); Tiffany L. Halo, Cambridge, MA (US); Aleksandar Filip Radovic-Moreno, State College, PA (US); Clayton Rische, Skokie, IL (US); Sagar Anantatmula, Skokie, IL (US)

(73) Assignee: Exicure, Inc, Skokie, IL (US)

(21) Appl. No.: 14/907,455

(22) PCT Filed: Jul. 25, 2014
(86) PCT No.: PCT/US2014/048294

§ 371 Date: Jan. 25, 2016

Related U.S. Application Data
(60) Provisional application No. 61/858,584, filed on Jul. 25, 2013.

(51) Int. Cl. C12N 15/117; A61K 47/48; A61K 39/39

(52) U.S. Cl. CPC C12N 15/117; A61K 39/39; A61K 47/48861; C12N 2320/30; C12N 2310/14; C12N 2310/17; C12N 2310/315; C12N 2310/351; C12N 2310/3515; A61K 2039/55561

(57) ABSTRACT

Aspects of the invention relate to nanoscale constructs and related methods and compositions thereof. The compositions of the invention are useful for treating disorders that are sensitive to levels of immune cell activation, such as autoimmune disease or other inflammation based disease or disorder.

### RELATED APPLICATIONS

This application claims priority under 35 U.S.C. §119(e) to U.S. Provisional Application Ser. No. 61/858,584, entitled “SPHERICAL NUCLEIC ACID-BASED CONSTRUCTS AS IMMUNOSTIMULATORY AGENTS FOR PROPHYLACTIC AND THERAPEUTIC USE,” filed on Jul. 25, 2013, which is herein incorporated by reference in its entirety.

### FIELD OF INVENTION

The invention relates to nanoscale constructs for delivering antagonists of nucleic acid-interacting complexes as well as methods and compositions thereof.

### BACKGROUND OF INVENTION

Immune cells, specifically macrophages, dendritic cells and B-cells, use Toll-like Receptors (TLRs) to survey the environment for foreign material such as bacterial components and foreign DNA or RNA⁴⁻¹⁰. Upon activation of these receptors a large inflammatory response is generated through specific cell signaling pathways, primarily through the transcription factor, NFκB¹¹. NFkB activation then results in the production of several secreted signaling molecule such as TNFα that promote the inflammatory response to neighboring immune cells¹¹. This immunological sensory system is referred to as the innate immune response. In several autoimmune disorders, the body incorrectly recognizes self-components such as DNA and RNA as foreign and will mount a massive inflammatory response that can be destructive, painful and life threatening if not controlled. The most prevalent examples of this include rheumatoid arthritis which attacks primarily the joints and can destroy cartilage and bone if not carefully regulated as well as Lupus which will also attack internal organs such as the heart, kidney, and lungs and can be fatal if not controlled. Current therapies rely mainly on sequestering the resultant cytokine production from immune cells through TNFa-binding antibodies (etanercept, etc.) as well as general immunosuppression with chemotherapeutic agents such as methotrexate and elimination of B-cell population to stave off adaptive immune responses. However, these treatments are often poorly tolerated and/or become susceptible to resistance acquisition over time. Therefore, development of an antagonist toward the activation of TLRs at the beginning of this signaling cascade should be a potent therapy to blockade the inappropriate recognition of self-DNA and RNA in patients suffering from autoimmune disorders.

TLRs 7, 8, and 9 are all resident with the endosome of immune cells. TLR9 recognizes unmethylated CpG motifs that are common to bacterial DNA but not human DNA^{5,10}. TLRs 7 and 8 both recognize a specific sequence of short single stranded RNA common to viral infections⁶⁻⁸. Importantly, mimics of these common recognition motifs that can antagonize their respective TLRs and block downstream signaling are known¹²⁻¹⁷. However, their use in therapies is limited due to their ability to be delivered to the sites of pathology without being degraded in vivo.

### SUMMARY OF INVENTION

Described herein are novel methods and compositions for regulating immune responses through the modulation of receptor interactions, such as TLRs, using a nanoscale construct. Aspects of the invention relate to a nanoscale construct having a corona of an antagonist of nucleic acid-interacting complex wherein the surface density of the antagonist of nucleic acid-interacting complexes is at least 0.3 pmol/cm².

In other aspects the invention is a nanoscale construct having a corona of an antagonist of nucleic acid-interacting complex, and an antigen incorporated into the corona. In some embodiments the surface density of the antigen is at least 0.3 pmol/cm². In other embodiments the antigen includes at least two different types of antigen.

In yet other aspects, the invention is a nanoscale construct having a corona with at least two antagonists of nucleic acid-interacting complexes incorporated, wherein the antagonists are selected from the group consisting of TLR 3, 7/8, and/or 9 antagonists.

In some embodiments the antagonist of nucleic acid-interacting complexes contains a spacer.

In other embodiments the antagonist of nucleic acid-interacting complexes is RNA or DNA. The antagonists of nucleic acid-interacting complexes may be, for instance, a double stranded RNA or double stranded DNA. Alternatively the antagonist of nucleic acid-interacting complexes may be a single stranded RNA. In some embodiments the antagonist of nucleic acid-interacting complexes is an unmethylated deoxyribonucleic acid, such as an optimized immunoregulatory sequence.

In certain embodiments, the nanoscale construct includes a nanoparticle core and optionally the nanoparticle core is metallic. In certain embodiments, the metal is selected from the group consisting of gold, silver, platinum, aluminum, palladium, copper, cobalt, indium, nickel and mixtures thereof. In certain embodiments, the nanoparticle core comprises gold. In certain embodiments, the nanoparticle core is a lattice structure including degradable gold. In some embodiments the nanoscale construct is degradable.

In certain embodiments, the diameter of the nanoscale construct is from 1 nm to about 250 nm in mean diameter, about 1 ran to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

In other aspects the invention is a nanoscale construct having a spherical corona of an antagonist of nucleic acid-interacting complexes, wherein the antagonist is nucleic acid having at least one phosphodiester internucleotide linkage. In some embodiments each internucleotide linkage of the nucleic acid is a phosphodiester linkage.

In some embodiments of the invention the corona is a spherical corona.

A vaccine composed of a nanoscale construct as described herein and a carrier is provided in other aspects of the invention.

A method for delivering a therapeutic agent to a cell by delivering the nanoscale construct of the invention to the cell is provided in other aspects.

A method for regulating expression of a target molecule is provided in other aspects of the invention. The method involves delivering the nanoscale construct of the invention to the cell. In some embodiments the target molecule is a TLR selected from the group consisting of TLR3, 7, 8, and 9.

A method for antagonizing a TLR by delivering the nanoscale construct as described herein to the cell is provided in other aspects of the invention.

According to other aspects the invention is a method of treating a subject, involving administering to the subject the nanoscale construct as described herein in an effective amount to reduce an immune response. In some embodiments the subject has an infectious disease, a cancer, an autoimmune disease, asthma, or an allergic disease, an inflammatory disease, a metabolic disease, a cardiovascular disease, or is a candidate for or the recipient of tissue or organ transplant.

In other aspects the invention is a method of modulating an immune response in a subject, by administering to the subject a nanoscale construct of a corona of an antagonist of nucleic acid-interacting complexes, wherein the antagonist is nucleic acid having at least one phosphodiester internucleotide linkage in an effective amount to modulate an immune response.

In certain embodiments, the method involves delivering a therapeutic or detection modality to a cell.

Further aspects of the invention relate to a kit comprising: a nanoparticle core; an antagonist and instructions for assembly of an antagonist-nanoparticle. In certain embodiments, the kit further comprises instructions for use.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1D are a set of graphs demonstrating that a construct of the invention (AST-015) is able to repress CpG induced TLR9 activation in macrophage-like RAW Blue cells. FIG. 1A shows AST-012, FIG. 1B shows AST-013, FIG. 1C shows AST-014, and FIG. 1D shows AST-015.

FIGS. 2A-2D are a set of graphs demonstrating that pre-treatment with a construct of the invention (AST-015) is able to repress CpG-induced TLR9 activation in macrophage-like RAW-Blue cells. Cells were incubated with the immunoregulatory constructs prior to stimulation with TLR9 agonists. IC50 values are presented in nanomolar (nM). The “Untreated” line refers to the TLR activation level of cells that never saw any stimulant.

FIGS. 3A-3D are a set of graphs demonstrating that simultaneous treatment with a construct of the invention (AST-015) and CpG DNA is able to repress CpG-induced TLR9 activation in macrophage-like RAW-Blue cells. The efficacies of free immunoregulatory DNA (FIGS. 3A and 3B) and immunoregulatory SNAs (FIGS. 3C and 3D) were compared in the RAW-Blue reporter cell line for TLR activation. Under these conditions, cells were incubated with the immunoregulatory constructs at the same time as stimulation with TLR9 agonists. IC50 values are presented in nanomolar (nM). The “Untreated” line refers to the TLR activation level of cells that never saw any stimulant.

FIG. 4 is a graph demonstrating that a construct of the invention (AST-015) is able to repress CpG-induced TLR9 activity in chronically stimulated macrophage-like RAW-Blue cells. The efficacies of free immunoregulatory DNA were determined in the RAW-Blue reporter cell line for TLR activation. Under these conditions, cells were first pre-stimulated with TLR9 agonists constructs to a chronic level and then incubated with the immunoregulatory constructs at the same time as re-stimulation with TLR9 agonists. IC50 values are presented in nanomolar (nM). The “Untreated” line refers to the TLR activation level of cells that never saw any stimulant. The “o/n Untreated” line refers to cells that saw stimulant overnight but did not receive a second dose of stimulant the following day.

FIGS. 5A and 5B are a set of graphs demonstrating that AST developed immunoregulatory sequence 4084F7/8 is able to repress both CpG-induced TLR9 activity and ssRNA-induced TLR7/8 activity in macrophage-like RAW-Blue cells. The 4084F sequence used in AST-015 and a modified 4084F7/8 sequence developed at AST were compared to clinical examples from Dynavax, IRS869 and IRS954, for efficacy against TLR9 (FIG. 5A) and TLR7/8 (FIG. 5B) agonists. IC50 values are presented in nanomolar (nM). The “Untreated” line refers to the TLR activation level of cells that never saw any stimulant.

FIG. 6 show a representation of a novel construct containing immunoregulatory DNA (irDNA). FIG. 6 shows that irSNAs may be synthesized using a 13 nm diameter gold nanoparticle as a template for the addition or thiolated irDNA and short ethylene glycol polymers.

FIGS. 7A-7D are graphs depicting the ability of the constructs of the invention to block a variety of agonists. Both tested constructs were able to block stimulation by all three agonists tested: imiquimod (TLR7, FIG. 7B), CpG 1826 (CpG, TLR9, FIG. 7D), bacterial lipopolysaccharide (LPS, TLR4, FIG. 7C), or all three simultaneously (FIG. 7B).

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

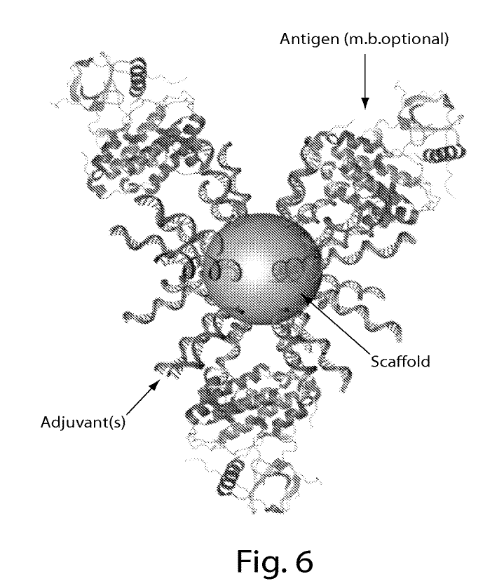

The present invention, in some aspects, relates to novel constructs or particles containing immunoregulatory DNA (irDNA). The immunoregulatory DNA may be, for example, TLR9, TLR7/8, and/or TLR7/8/9 antagonistic DNA oligonucleotides. The particles have a dense arrangement of oligonucleotide structures adhered thereto, which are referred to herein, equivalently, as nanoparticle constructs, nanoscale constructs or irSNAs. These constructs are capable of antagonizing TLR-mediated signaling in response to non-methylated CpG-containing single stranded oligonucleotides and single stranded RNA agonists common to several autoimmune pathologies. Some exemplary data is presented in the examples below. In this data it is shown that irSNAs may be synthesized using a 13 nm diameter gold nanoparticle as a template for the addition or thiolated irDNA and short ethylene glycol polymers (shown in Scheme 1, FIG. 6B). It was discovered that irSNAs containing irDNAs against endosomally resident TLRs are able to provide a potent and novel approach to deliver irDNA to immune cells to block over-activation of TLR-mediated signaling pathways common to disorders such as autoimmune disorders such as Rheumatoid Arthritis. The discovery that these constructs were significantly more effective than existing methods for delivering irDNA for the treatment of disorders, was quite unexpected. Although Applicant is not bound by a mechanism it is believed that the density of the irDNA as it is presented in the constructs of the invention greatly enhance the nucleic acid receptor modulation.

The data presented in the Examples demonstrate that irSNAs are a potent inhibitor of TLR9 and TLR7/8-mediated NFκB and TNFα immune activation signaling in murine macrophage-like cells (RAW). In one example, low nM doses of immunoregulatory oligonucleotide sequences incorporated in the irSNAs were capable of blocking activated TLR9- and TLR7/8-mediated NFκB/TNFα signaling. Importantly, DNA with natural phosphodiester backbones were efficacious when incorporated into irSNAs, but not when administered as free oligonucleotides in solution. irSNAs incorporating phosphorothioate (ps) backbone containing sequences were able to modulate TLR activation as effectively as free DNA administration, but having the added advantage of a longer release profile. Most current DNA-based therapies require phosphorothioate backbone modifications for any efficacy, but are limited in therapeutic window due to phosphorothioate-mediated general toxicity. The fact that the constructs of the invention can incorporate both natural and modified backbone chemistries greatly enhances the potential therapeutic window for therapies developed on this platform.

NFκB and TNFα signaling pathways are major contributors to the acute pathology of autoimmune disorders, specifically RA¹⁸. Traditional therapies rely mainly on sequestration strategies to down-regulate the effects of over-activated immune systems and are reactionary by nature¹⁹⁻²¹. irSNAs proactively regulate immune signaling by blocking the receptor that is mainly responsible for the cascade of signaling that results in activation of pro-inflammatory cellular responses. Employing this mechanism of action offers significant potential improvements in treating autoimmune patients, including RA patients, for instance resulting in enhanced potency, reduced chance of resistance, and the potential for greater therapeutic windows. Unlike many broad-spectrum treatments, since the irSNAs only block over activation of these signaling pathways that are primarily present only in immune cells, the side effects to normal tissues and cells may be reduced or even eliminated with the administration of irSNAs.

Aspects of the invention relate to nanoscale constructs. A nanoscale construct refers to a nanometer sized construct having one or more nucleic acids held in a geometrical position. The nanoscale construct typically is referred to as a corona of a set of nucleic acids. A corona, as used herein, refers to an exterior shell composed of nucleic acid molecules. The corona may have a nanoparticle core composed of nucleic acids or other materials, such as metals. Alternatively, the corona may simply be a set of nucleic acids arranged in a geometric shape with a hollow core, i.e. a 3-dimensionally shaped layer of nucleic acids. Typically, but not always, the corona has a spherical shape.

In the instance, when the corona includes a nanoparticle core the nucleic acids may be linked directly to the core. Some or all of the nucleic acids may be linked to other nucleic acids either directly or indirectly through a covalent or non-covalent linkage. The linkage of one nucleic acid to another nucleic acid may be in addition to or alternatively to the linkage of that nucleic acid to a core. One or more of the nucleic acids may also be linked to other molecules such as an antigen.

When the corona does not include a nanoparticle core, the nucleic acids may be linked to one another either directly or indirectly through a covalent or non-covalent linkage. In some embodiments the corona that does not include a nanoparticle core may be formed by layering the nucleic acids on a lattice or other dissolvable structure and then dissolving the lattice or other structure to produce an empty center.

As used herein, the nano scale construct is a construct having an average diameter on the order of nanometers (i.e., between about 1 nm and about 1 micrometer. For example, in some instances, the diameter of the nanoparticle is from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, about 1 nm to about 20 nm in mean diameter, about 1 nm to about 10 nm in mean diameter, about 5 nm to about 150 nm in mean diameter, about 5 to about 50 nm in mean diameter, about 10 to about 30 nm in mean diameter, about 10 to 150 nm in mean diameter, about 10 to about 100 nm in mean diameter, about 10 to about 50 nm in mean diameter, about 30 to about 100 nm in mean diameter, or about 40 to about 80 nm in mean diameter.

In some instances the corona includes a nanoparticle core that is attached to one or more antagonists of nucleic acid-interacting complexes and/or antigens. As used herein, a nanoparticle core refers to the nanoparticle component of a nanoparticle construct, without any attached modalities. In some instances, the nanoparticle core is metallic. It should be appreciated that the nanoparticle core can comprise any metal. Several non-limiting examples of metals include gold, silver, platinum, aluminum, palladium, copper, cobalt, indium, nickel and mixtures thereof. In some embodiments, the nanoparticle core comprises gold. For example, the nanoparticle core can be a lattice structure including degradable gold. Nanoparticles can also comprise semiconductor and magnetic materials.

Non-limiting examples of nanoparticles compatible with aspects of the invention are described in and incorporated by reference from: U.S. Pat. No. 7,238,472, US Patent Publication No. 2003/0147966, US Patent Publication No. 2008/0306016, US Patent Publication No. 2009/0209629, US Patent Publication No. 2010/0136682, US Patent Publication No. 2010/0184844, US Patent Publication No. 2010/0294952, US Patent Publication No. 2010/0129808, US Patent Publication No. 2010/0233270, US Patent Publication No. 2011/0111974, PCT Publication No. WO 2002/096262, PCT Publication No. WO 2003/08539, PCT Publication No. WO 2006/138145, PCT Publication No. WO 2008/127789, PCT Publication No. WO 2008/098248, PCT Publication No. WO 2011/079290, PCT Publication No. WO 2011/053940, PCT Publication No. WO 2011/017690 and PCT Publication No. WO 2011/017456. Nanoparticles associated with the invention can be synthesized according to any means known in the art or can be obtained commercially. For example, several non-limiting examples of commercial suppliers of nanoparticles include: Ted Pella, Inc., Redding, Calif., Nanoprobes, Inc., Yaphank, N.Y., Vacuum Metallurgical Co., Ltd., Chiba, Japan and Vector Laboratories, Inc., Burlington, Calif.

A nucleic acid-interacting complex as used herein refers to a molecule or complex of molecules that interact with a nucleic acid molecule and, for instance, are stimulated to produce an immune response in response to that interaction. The molecule or complex of molecules may be a receptor. In some embodiments a nucleic acid-interacting complex is a pattern recognition receptor (PRR) complex. PRRs are a primitive part of the immune system composed of proteins expressed by cells of the innate immune system to identify pathogen-associated molecular patterns (PAMPs), which are associated with microbial pathogens or cellular stress, as well as damage-associated molecular patterns (DAMPs), which are associated with cell components released during cell damage. PRRs include but are not limited to membrane-bound PRRs, such as receptor kinases, toll-like receptors (TLR), and C-type lectin Receptors (CLR) (mannose receptors and asialoglycoprotein receptors); Cytoplasmic PRRs such as RIG-I-like receptors (RLR), RNA Helicases, Plant PRRs, and NonRD kinases; and secreted PRRs.

Nucleic acid-interacting complexes include but are not limited to TLRs, RIG-I, transcription factors, cellular translation machinery, cellular transcription machinery, nucleic-acid acting enzymes, and nucleic acid associating autoantigens. Nucleic acid molecules that are antagonists of a nucleic acid-interacting complex include but are not limited to TLR antagonists and antagonists of RIG-I, transcription factors, cellular translation machinery, cellular transcription machinery, nucleic-acid acting enzymes, and nucleic acid associating autoantigens.

In some embodiments an antagonist of a nucleic acid-interacting complex is a TLR antagonist. A TLR antagonist, as used herein is a nucleic acid molecule that interacts with and modulates, i.e. reduces, the activity of a TLR.

Toll-like receptors (TLRs) are a family of highly conserved polypeptides that play a critical role in innate immunity in mammals. At least ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For a review, see Aderem A et al. (2000) Nature 406:782-87.

TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR7 are known. See, for example, GenBank Accession Nos. AF240467, AF245702, NM_016562, AF334942, NM_133211; and AAF60188, AAF78035, NP_057646, AAL73191, and AAL73192, the contents of all of which are incorporated herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

Nucleotide and amino acid sequences of human and murine TLR8 are known. See, for example, GenBank Accession Nos. AF246971, AF245703, NM_016610, XM_045706, AY035890, NM_133212; and AAF64061, AAF78036, NP_057694, XP_045706, AAK62677, and NP_573475, the contents of all of which is incorporated herein by reference. Human TLR8 is reported to exist in at least two isoforms, one 1041 amino acids long and the other 1059 amino acids long. Murine TLR8 is 1032 amino acids long. TLR8 polypeptides include an extracellular domain having a leucine-rich repeat region, a transmembrane domain, and an intracellular domain that includes a TIR domain.

Nucleotide and amino acid sequences of human and murine TLR9 are known. See, for example, GenBank Accession Nos. NM_017442, AF259262, AB045180, AF245704, AB045181, AF348140, AF314224, NM_031178; and NP_059138, AAF72189, BAB19259, AAF78037, BAB19260, AAK29625, AAK28488, and NP_112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

As used herein, the term “TLR signaling” refers to any aspect of intracellular signaling associated with signaling through a TLR. As used herein, the term “TLR-mediated immune response” refers to the immune response that is associated with TLR signaling. A reduction in TLR signaling or activity refers to a decrease in signaling or activity relative to baseline. A baseline level may be a level where an immunostimulatory molecule is causing stimulation of a TLR. In that instance a reduction in signaling or activity is a reduction in signaling or activity with respect to the level of signaling or activity achieved by the immunostimulatory molecule.

A TLR7-mediated immune response is a response associated with TLR7 signaling. TLR7-mediated immune response is generally characterized by the induction of IFN-α and IFN-inducible cytokines such as IP-10 and I-TAC. The levels of cytokines IL-1α/β, IL-6, IL-8, MIP-1α/β and MIP-3α/β induced in a TLR7-mediated immune response are less than those induced in a TLR8-mediated immune response.

A TLR8-mediated immune response is a response associated with TLR8 signaling. This response is further characterized by the induction of pro-inflammatory cytokines such as IFN-γ, IL-12p40/70, TNF-α, IL-1α/β, IL-6, IL-8, MIP-1α/β and MIP-3α/β.

A TLR9-mediated immune response is a response associated with TLR9 signaling. This response is further characterized at least by the production/secretion of IFN-γ and IL-12, albeit at levels lower than are achieved via a TLR8-mediated immune response.

As used herein, a “TLR7/8 antagonist” collectively refers to any nucleic acid that is capable of decreasing TLR7 and/or TLR8 signaling (i.e., an antagonist of TLR7 and/or TLR8) relative to a baseline level. Some TLR7/8 antagonists decrease TLR7 signaling alone (e.g., TLR7 specific antagonists), some decrease TLR8 signaling alone (e.g., TLR8 specific antagonists), and others decrease both TLR7 and TLR8 signaling.

As used herein, the term “TLR9 antagonist” refers to any agent that is capable of decreasing TLR9 signaling (i.e., an antagonist of TLR9).

In some embodiments antagonists of TLR 7, 8, or 9 include immunoregulatory nucleic acids. Immunoregulatory nucleic acids include but are not limited to nucleic acids falling within the following formulas: 5′RₙJGCN_{z}3′, wherein each R is a nucleotide, n is an integer from about 0 to 10, J is U or T, each N is a nucleotide, and z is an integer from about 1 to about 100. In some embodiments, ₙ is 0 and _{z} is from about 1 to about 50. In some embodiments N is 5′S₁S₂S₃S₄3′, wherein S₁, S₂, S₃, and S₄ are independently G, I, or 7-deaza-dG. In some embodiments the TLR7 TLR8 and/or TLR9 antagonist is selected from the group consisting of TCCTGGAGGGGTTGT (SEQ ID NO: 1), TGCTCCTGGAGGGGTTGT (SEQ ID NO: 2), TGCTGGATGGGAA (SEQ ID NO: 3), TGCCCTGGATGGGAA (SEQ ID NO: 4), TGCTTGACACCTGGATGGGAA (SEQ ID NO: 5), TGCTGGATGGGAA/iSp18//iSp18//3ThioMC3-D/(13nm AuNP; SEQ ID NO: 6), TGCCCTGGATGGGAA/iSp18//iSp18//3ThioMC3-D/(13nm AuNP; SEQ ID NO: 7), TGCTTGACACCTGGATGGGAA/iSp18//iSp18//3ThioMC3-D/(13nm AuNP; SEQ ID NO: 8), TCCTGAGCTTGAAGT/iSp18//iSp18//3ThioMC3-D/ (SEQ ID NO: 9), TCCTGAGCTTGAAGT/iSp18//iSp18//3ThioMC3-D/(13nm AuNP; SEQ ID NO: 10), TTCTGGCGGGGAAGT/iSp18//iSp18//3ThioMC3-D/ (SEQ ID NO: 11), CTCCTATTGGGGGTTTCCTAT/iSp18//iSp18//3ThioMC3-D/ (SEQ ID NO: 12), ACCCCCTCTACCCCCTCTACCCCTCT/iSp18//iSp18//3ThioMC3-D/ (SEQ ID NO: 13), CCTGGATGGGAA/iSp18//iSp18//3ThioMC3-D/ (SEQ ID NO: 14), TTCTGGCGGGGAAGT/iSp18//iSp18//3ThioMC3-D/(13nm AuNP; SEQ ID NO: 15), CTCCTATTGGGGGTTTCCTAT/iSp18//iSp18//3ThioMC3-D/(13nm AuNP; SEQ ID NO: 16), ACCCCCTCTACCCCCTCTACCCCTCT/iSp18//iSp18//3ThioMC3-D/(13nm AuNP; SEQ ID NO: 17), CCTGGATGGGAA/iSp18//iSp18//3ThioMC3-D/(13nm AuNP; SEQ ID NO: 18), C*C*T*GGATGGGAA/iSp18//iSp18//3ThioMC3-D/(13nm AuNP; SEQ ID NO: 19), CCTGGATG*G*G*AA/iSp18//iSp18//3ThioMC3-D/(13nm AuNP; SEQ ID NO: 20), C*C*T*GGATG*G*G*AA/iSp18//iSp18//3ThioMC3-D/(13nm AuNP; SEQ ID NO: 21), /Chol/CCTGGATGGGAA/iSp18//iSp18//3ThioMC3-D/(13nm AuNP; SEQ ID NO: 22), /Stryl/CCTGGATGGGAA/iSp18//iSp18//3ThioMC3-D/(13nm AuNP; SEQ ID NO: 23), /Palm/CCTGGATGGGAA/iSp18//iSp18//3ThioMC3-D/(13nm AuNP; SEQ ID NO: 24)
T*C*C*T*G*G*A*G*G*G*G*T*T*G*T (SEQ ID NO: 25)
T*G*C*T*C*C*T*G*G*A*G*G*G*G*T*T*G*T (SEQ ID NO: 26)
T*G*C*T*G*G*A*T*G*G*G*A*A (SEQ ID NO: 27)
T*G*C*C*C*T*G*G*A*T*G*G*G*A*A (SEQ ID NO: 28)
T*G*C*T*T*G*A*C*A*C*C*T*G*G*A*T*G*G*G*A*A (SEQ ID NO: 29)
T*G*C*T*G*G*A*T*G*G*G*A*A*/iSp18//iSp18//3ThioMC3-D/(13nm AuNP; SEQ ID NO: 30)
T*G*C*C*C*T*G*G*A*T*G*G*G*A*A*/iSp18//iSp18//3ThioMC3-D/(13nm AuNP; SEQ ID NO: 31)
T*G*C*T*T*G*A*C*A*C*C*T*G*G*A*T*G*G*G*A*A*/iSp18//iSp18//3ThioMC3-D/(13nm AuNP; SEQ ID NO: 32)
T*C*C*T*G*A*G*C*T*T*G*A*A*G*T*/iSp18//iSp18//3ThioMC3-D/(13nm AuNP; SEQ ID NO: 33)
T*C*C*T*G*A*G*C*T*T*G*A*A*G*T*/iSp18//iSp18//3ThioMC3-D/(13nm AuNP; SEQ ID NO: 34)
T*T*C*T*G*G*C*G*G*G*G*A*A*G*T*/iSp18//iSp18//3ThioMC3-D/(13nm AuNP; SEQ ID NO: 35)
C*T*C*C*T*A*T*T*G*G*G*G*G*T*T*T*C*C*T*A*T*/iSp18//iSp18//3ThioMC3-D/(13nm AuNP; SEQ ID NO: 36)
A*C*C*C*C*C*T*C*T*A*C*C*C*C*C*T*C*T*A*C*C*C*C*T*C*T*/iSp18//iSp18//3Thio MC3-D/(13nm AuNP; SEQ ID NO: 37)
C*C*T*G*G*A*T*G*G*G*A*A*/iSp18//iSp18//3ThioMC3-D/(13nm AuNP; SEQ ID NO: 38)
T*T*C*T*G*G*C*G*G*G*G*A*A*G*T*/iSp18//iSp18//3ThioMC3-D/(13nm AuNP; SEQ ID NO: 39)
C*T*C*C*T*A*T*T*G*G*G*G*G*T*T*T*C*C*T*A*T*/iSp18//iSp18//3ThioMC3-D/(13nm AuNP; SEQ ID NO: 40)
A*C*C*C*C*C*T*C*T*A*C*C*C*C*C*T*C*T*A*C*C*C*C*T*C*T*/iSp18//iSp18//3Thio MC3-D/(13nm AuNP; SEQ ID NO: 41)
C*C*T*G*G*A*T*G*G*G*A*A*/iSp18//iSp18//3ThioMC3-D/(13nm AuNP; SEQ ID NO: 42)
(13nm AuNP)/5ThioMC3-D//iSp18//iSp18/*T*G*C*T*G*G*A*T*G*G*G*A*A (13nm AuNP; SEQ ID NO: 43)
/5ThioMC3-D//iSp18//iSp18/*T*G*C*C*C*T*G*G*A*T*G*G*G*A*A (SEQ ID NO:44)
(13nm AuNP)/5ThioMC3-D//iSp18//iSp18/*T*G*C*T*T*G*A*C*A*C*C*T*G*G*A*T*G*G*G*A*A (SEQ ID NO: 45)
(13nm AuNP)/5ThioMC3-D//iSp18//iSp18/*T*C*C*T*G*A*G*C*T*T*G*A*A*G*T (SEQ ID NO: 46)
(13nm AuNP)/5ThioMC3-D//iSp18//iSp18/*T*C*C*T*G*A*G*C*T*T*G*A*A*G*T (SEQ ID NO: 47)
(13nm AuNP)/5ThioMC3-D//iSp18//iSp18/*T*T*C*T*G*G*C*G*G*G*G*A*A*G*T (SEQ ID NO: 48)
(13nm AuNP)/5ThioMC3-D//iSp18//iSp18/*C*T*C*C*T*A*T*T*G*G*G*G*G*T*T*T*C*C*T*A*T (SEQ ID NO: 49)
(13nm AuNP)/5ThioMC3-D//iSp18//iSp18/*A*C*C*C*C*C*T*C*T*A*C*C*C*C*C*T*C*T*A*C*C*C*C*T*C*T (SEQ ID NO: 50)
(13nm AuNP)/5ThioMC3-D//iSp18//iSp18/*C*C*T*G*G*A*T*G*G*G*A*A (SEQ ID NO: 51)
(13nm AuNP)/5ThioMC3-D//iSp18//iSp18/*T*T*C*T*G*G*C*G*G*G*G*A*A*G*T (SEQ ID NO: 52)
(13nm AuNP)/5ThioMC3-D//iSp18//iSp18/*C*T*C*C*T*A*T*T*G*G*G*G*G*T*T*T*C*C*T*A*T (SEQ ID NO: 53)
(13nm AuNP)/5ThioMC3-D//iSp18//iSp18/*A*C*C*C*C*C*T*C*T*A*C*C*C*C*C*T*C*T*A*C*C*C*C*T*C*T (SEQ ID NO: 54)
(13nm AuNP)/5ThioMC3-D//iSp18//iSp18/*C*C*T*G*G*A*T*G*G*G*A*A (SEQ ID NO: 55) TTAGGGTTAGGGTTAGGGTTAGGG (SEQ ID NO: 56)
T*T*A*G*G*G*T*T*A*G*G*G*T*T*A*G*G*G*T*T*A*G*G*G (SEQ ID NO: 57)
TTAGGGTTAGGGTTAGGGTTAGGG (SEQ ID NO: 58)/iSp18//iSp18//3ThioMC3-D/(13nm AuNP)
T*T*A*G*G*G*T*T*A*G*G*G*T*T*A*G*G*G*T*T*A*G*G*G* (SEQ ID NO: 59)/iSp18//iSp18//3ThioMC3-D/(13nm AuNP)
(13nm AuNP)/5ThioMC3-D//iSp18//iSp18/TTAGGGTTAGGGTTAGGGTTAGGG (SEQ ID NO: 60)
(13nm AuNP)/5ThioMC3-D//iSp18//iSp18/*T*T*A*G*G*G*T*T*A*G*G*G*T*T*A*G*G*G*T*T*A*G*G*G (SEQ ID NO: 61)
CTATCTGUCGTTCTCTGU (SEQ ID NO: 62)
C*T*A*T*C*T*G*U*C*G*T*T*C*T*C*T*G*U (SEQ ID NO: 63)
CTATCTGUCGTTCTCTGU (SEQ ID NO: 64)/iSp18//iSp18//3ThioMC3-D/(13nm AuNP)
C*T*A*T*C*T*G*U*C*G*T*T*C*T*C*T*G*U*(SEQ ID NO: 65)/iSp18//iSp18//3ThioMC3-D/(13nm AuNP)
(13nm AuNP)/5ThioMC3-D//iSp18//iSp18/CTATCTGUCGTTCTCTGU (SEQ ID NO: 66)
(13nm AuNP)/5ThioMC3-D//iSp18//iSp18/*C*T*A*T*C*T*G*U*C*G*T*T*C*T*C*T*G*U (SEQ ID NO: 67)
(13nm AuNP)/5ThioMC3-D//iSp18//iSp18/TTAGGGTTAGGGTTAGGGTTAGGG (SEQ ID NO: 68)
(13nm AuNP)/5ThioMC3-D//iSp18//iSp18/T*T*A*G*G*G*T*T*A*G*G*G*T*T*A*G*G*G*T*T*A*G*G*G* (SEQ ID NO: 69)
(13nm AuNP)/5ThioMC3-D//iSp18//iSp18/CTATCTGUCGTTCTCTGU (SEQ ID NO: 70)
(13nm AuNP)/5ThioMC3-D//iSp18//iSp18/C*T*A*T*C*T*G*U*C*G*T*T*C*T*C*T*G*U*(SEQ ID NO: 71)
(13nm AuNP)/5ThioMC3-D//iSp18//iSp18/TGCTGGATGGGAA (SEQ ID NO: 72)
(13nm AuNP)/5ThioMC3-D//iSp18//iSp18/TGCCCTGGATGGGAA (SEQ ID NO: 73)
(13nm AuNP)/5ThioMC3-D//iSp18//iSp18/TGCTTGACACCTGGATGGGAA (SEQ ID NO: 74)
(13nm AuNP)/5ThioMC3-D//iSp18//iSp18/TCCTGAGCTTGAAGT (SEQ ID NO: 75)
(13nm AuNP)/5ThioMC3-D//iSp18//iSp18/TCCTGAGCTTGAAGT (SEQ ID NO: 76)
(13nm AuNP)/5ThioMC3-D//iSp18//iSp18/TTCTGGCGGGGAAGT (SEQ ID NO: 77)
(13nm AuNP)/5ThioMC3-D//iSp18//iSp18/CTCCTATTGGGGGTTTCCTAT (SEQ ID NO: 78)
(13nm AuNP)/5ThioMC3-D//iSp18//iSp18/ACCCCCTCTACCCCCTCTACCCCTCT (SEQ ID NO: 79)
(13nm AuNP)/5ThioMC3-D//iSp18//iSp18/CCTGGATGGGAA (SEQ ID NO: 80)
(13nm AuNP)/5ThioMC3-D//iSp18//iSp18/TTCTGGCGGGGAAGT (SEQ ID NO: 81)
(13nm AuNP)/5ThioMC3-D//iSp18//iSp18/CTCCTATTGGGGGTTTCCTAT (SEQ ID NO: 82)
(13nm AuNP)/5ThioMC3-D//iSp18//iSp18/ACCCCCTCTACCCCCTCTACCCCTCT (SEQ ID NO: 83)
(13nm AuNP)/5ThioMC3-D//iSp18//iSp18/CCTGGATGGGAA (SEQ ID NO: 84)
(13nm AuNP)/5ThioMC3-D//iSp18//iSp18/C*C*T*GGATGGGAA (SEQ ID NO: 85)
(13nm AuNP)/5ThioMC3-D//iSp18//iSp18/CCTGGATG*G*G*AA (SEQ ID NO: 86)
(13nm AuNP)/5ThioMC3-D//iSp18//iSp18/C*C*T*GGATG*G*G*AA (SEQ ID NO: 87)
(13nm AuNP)/5ThioMC3-D//iSp18//iSp18/CCTGGATGGGAA/Chol/ (SEQ ID NO: 88)
(13nm AuNP)/5ThioMC3-D//iSp18//iSp18/CCTGGATGGGAA/Stryl/ (SEQ ID NO: 89) and
(13nm AuNP)/5ThioMC3-D//iSp18//iSp18/CCTGGATGGGAA/Palm/ (SEQ ID NO: 90).

In some embodiments the antagonists of nucleic acid-interacting complexes are described in references 23 and 24, each of which is incorporated by reference.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). A polynucleotide of the nanoscale construct and optionally attached to a nanoparticle core can be single stranded or double stranded. A double stranded polynucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded polynucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded polynucleotide of the invention contains at least or up to 1, 2, 3, 4, 5, 6, 7, 8, 9, 10, 11, 12, 13, 14, or 15 mismatches.

Polynucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR₂,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In some embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “hydrophobic modifications’ refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O⁻ (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the nucleomonomers of a polynucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²⁻)—O—) that covalently couples adjacent nucleomonomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

In some aspects, polynucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, polynucleotides can comprise both DNA and RNA.

In some aspects, at least a portion of the contiguous polynucleotides are linked by a substitute linkage, e.g., a phosphorothioate linkage. The presence of substitute linkages can improve pharmacokinetics due to their higher affinity for serum proteins.

The oligonucleotides of the nanoscale construct are preferably in the range of 6 to 100 bases in length. However, nucleic acids of any size greater than 6 nucleotides (even many kb long) are capable of inducing an immune response according to the invention if sufficient immunoregulatory motifs are present. Preferably the nucleic acid is in the range of between 8 and 100 and in some embodiments between 8 and 50 or 8 and 30 nucleotides in size.

In some embodiments the immunoregulatory oligonucleotides have a modified backbone such as a phosphorothioate (PS) backbone. In other embodiments the immunoregulatory oligonucleotides have a phosphodiester (PO) backbone. In yet other embodiments immunoregulatory oligonucleotides have a mixed PO and PS backbone.

Modalities associated with the invention, including antagonists of nucleic acid-interacting complexes and antigens, can be attached to nanoparticle cores by any means known in the art. Methods for attaching oligonucleotides to nanoparticles are described in detail in and incorporated by reference from US Patent Publication No. 2010/0129808.

A nanoparticle can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, polynucleotides are attached to nanoparticles by terminating the polynucleotide with a 5′ or 3′ thionucleoside. In other instances, an aging process is used to attach polynucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123.

In some instances, the nucleic acid and/or antigen are covalently attached to the nanoparticle core, such as through a gold-thiol linkage. A spacer sequence can be included between the attachment site and the uptake control moiety and/or the binding moiety. In some embodiments, a spacer sequence comprises or consists of an oligonucleotide, a peptide, a polymer or an oligoethylene.

Nanoscale constructs can be designed with multiple chemistries. For example, a DTPA (dithiol phosphoramidite) linkage can be used. The DTPA resists intracellular release of flares by thiols and can serve to increase signal to noise ratio.

The conjugates produced by the methods described herein are considerably more stable than those produced by other methods. This increased stability is due to the increased density of the oligonucleotides on the surfaces of a nanoparticle core or forming the surface of the corona. By performing the salt additions in the presence of a surfactant, for example approximately 0.01% sodium dodecylsulfate (SDS), Tween, or polyethylene glycol (PEG), the salt aging process can be performed in about an hour.

The surface density may depend on the size and type of nanoparticles and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 10 picomoles/cm will be adequate to provide stable nanoparticle-oligonucleotide conjugates. Preferably, the surface density is at least 15 picomoles/cm. Since the ability of the oligonucleotides of the conjugates to hybridize with targets may be diminished if the surface density is too great, the surface density optionally is no greater than about 35-40 picomoles/cm². Methods are also provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm, at least 50 pmol/cm², or 50 pmol/cm² or more.

Aspects of the invention relate to delivery of nanoscale constructs to a subject for therapeutic and/or diagnostic use. The particles may be administered alone or in any appropriate pharmaceutical carrier, such as a liquid, for example saline, or a powder, for administration in vivo. They can also be co-delivered with larger carrier particles or within administration devices. The particles may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, nanoscale constructs associated with the invention are mixed with a substance such as a lotion (for example, aquaphor) and are administered to the skin of a subject, whereby the nanoscale constructs are delivered through the skin of the subject. It should be appreciated that any method of delivery of nanoparticles known in the art may be compatible with aspects of the invention.

For use in therapy, an effective amount of the particles can be administered to a subject by any mode that delivers the particles to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

Thus, the invention in one aspect involves the finding that antagonists of nucleic acid-interacting complexes are highly effective in mediating immune modulatory effects. These antagonists of nucleic acid-interacting complexes are useful therapeutically and prophylactically for modulating the immune system to treat cancer, infectious diseases, allergy, asthma, autoimmune disease, and other inflammatory based diseases.

According to other aspects the invention is a method of treating a subject, involving administering to the subject the nanoscale construct as described herein in an effective amount to reduce an immune response. In some embodiments the subject has an infectious disease, a cancer, an autoimmune disease, asthma, or an allergic disease, an inflammatory disease, a metabolic disease, a cardiovascular disease, or is a candidate for or the recipient of tissue or organ transplant.

Examples of metabolic diseases include, but are not limited to, Type I diabetes, disorders of carbohydrate metabolism, amino acid metabolism, organic acid metabolism, fatty acid oxidation and mitochondrial metabolism, prophyrin metabolism, purine or pyrimidine metabolism, steroid metabolism, lysosomal mitochondrial function, peroxisomal function, lysosomal storage, urea cycle disorders (e.g., N-acetyl glutamate synthetase deficiency, carbamylphosphate synthase deficiency, ornithine carbamyl transferase deficiency, crginosuccinic aciduria, citrullinaemia, arginase deficiency), amino acid disorders (e.g., Non-ketotic hyperglycinaemia, tyrosinaemia (Type I), Maple syrup urine disease), organic acidemias (e.g, isovaleric acidemia, methylmalonic acidemia, propionic acidemia, glutaric aciduria type I, glutaric acidemia type I & II), mitochondrial disorders (e.g., carboxylase defects, mitochondrial myopathies, lactic acidosis (pyruvate dehydrogenase complex defects), congenital lactic acidosis, mitochondrial respiratory chain defects, cystinosis, Gaucher's disease, Fabry's disease, Pompe's disease, mucopolysaccharoidosis I, mucopolysaccharoidosis II, mucopolysaccharoidosis VI).

Cardiovascular disease refers to a number of disorders of the heart and vascular system. Typically, the cardiovascular disease is selected from the group comprising: cardiac hypertrophy; myocardial infarction; stroke; arteriosclerosis; and heart failure. In some instances the cardiovascular disease is associated with inflammation such as inflammation associated with atherosclerosis.

The antagonists of nucleic acid-interacting complexes useful in some aspects of the invention as a vaccine for the treatment of a subject at risk of developing or a subject having allergy or asthma, an infection with an infectious organism or a cancer in which a specific cancer antigen has been identified. In this instance, the vaccines may be tolorigenic vaccines. These may be administered with an immunosuppresant. It is particularly useful for the treatment of allergy, allergic disease and autoimmune disease. The antagonists of nucleic acid-interacting complexes can also be given without the antigen or allergen for protection against infection, allergy or cancer, and in this case repeated doses may allow longer term protection. A subject at risk as used herein is a subject who has any risk of exposure to an infection causing pathogen or a cancer or an allergen or a risk of developing cancer.

A subject having an infection is a subject that has been exposed to an infectious pathogen and has acute or chronic detectable levels of the pathogen in the body. The immunoregulatory oligonucleotides can be used to reduce an immune response associated with the infection. It is particularly desirable when a subject is at risk of developing sepsis. The constructs of the invention are useful for preventing aberrant responses associated with infection such as sepsis.

A subject having an allergy is a subject that has or is at risk of developing an allergic reaction in response to an allergen. An allergy refers to acquired hypersensitivity to a substance (allergen). Allergic conditions include but are not limited to eczema, allergic rhinitis or coryza, hay fever, conjunctivitis, bronchial asthma, urticaria (hives) and food allergies, and other atopic conditions.

A subject having a cancer is a subject that has detectable cancerous cells. In particular the cancer is a cancer associated with chronic inflammation. For instance cancers associated with inflammation caused by infection or to conditions such as chronic inflammatory bowel disease are associated with a significant number of cancers. Some triggers of chronic inflammation that increase cancer risk or progression include infections (e.g. Helicobacter pylori for gastric cancer and mucosal lymphoma; papilloma virus and hepatitis viruses for cervical and liver carcinoma, respectively), autoimmune diseases (e.g. inflammatory bowel disease for colon cancer) and inflammatory conditions of uncertain origin (e.g. prostatitis for prostate cancer). The constructs of the invention assist in controlling the chronic inflammation, reducing the triggers for causing or aggravating the cancer.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors, infections, and allergy/asthma in non-human subjects.

As used herein, the term treat, treated, or treating when used with respect to an disorder such as an infectious disease, cancer, allergy, or asthma refers to a prophylactic treatment which increases the resistance of a subject to development of the disease (e.g., to infection with a pathogen) or, in other words, decreases the likelihood that the subject will develop the disease (e.g., become infected with the pathogen) as well as a treatment after the subject has developed the disease in order to fight the disease (e.g., reduce or eliminate the infection) or prevent the disease from becoming worse.

An antigen as used herein is a molecule capable of provoking an immune response. Antigens include but are not limited to cells, cell extracts, proteins, polypeptides, peptides, polysaccharides, polysaccharide conjugates, peptide and non-peptide mimics of polysaccharides and other molecules, small molecules, lipids, glycolipids, carbohydrates, viruses and viral extracts and multicellular organisms such as parasites and allergens. The term antigen broadly includes any type of molecule which is recognized by a host immune system as being foreign. Antigens include but are not limited to cancer antigens, microbial antigens, and allergens.

A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

A microbial antigen as used herein is an antigen of a microorganism and includes but is not limited to virus, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

An allergen refers to a substance (antigen) that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

The nanoscale constructs of the invention may also be coated with or administered in conjunction with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics. Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

As used herein, the terms “cancer antigen” and “tumor antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

The antagonists of nucleic acid-interacting complexes are also useful for treating and preventing autoimmune disease. Autoimmune disease is a class of diseases in which an subject's own antibodies react with host tissue or in which immune effector T cells are autoreactive to endogenous self-peptides and cause destruction of tissue. Thus an immune response is mounted against a subject's own antigens, referred to as self-antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

A “self-antigen” as used herein refers to an antigen of a normal host tissue. Normal host tissue does not include cancer cells. Thus an immune response mounted against a self-antigen, in the context of an autoimmune disease, is an undesirable immune response and contributes to destruction and damage of normal tissue, whereas an immune response mounted against a cancer antigen is a desirable immune response and contributes to the destruction of the tumor or cancer. Thus, in some aspects of the invention aimed at treating autoimmune disorders it is not recommended that the immunoregulatory nucleic acids be administered with self-antigens, particularly those that are the targets of the autoimmune disorder.

In other instances, the immunoregulatory nucleic acids may be delivered with low doses of self-antigens. A number of animal studies have demonstrated that mucosal administration of low doses of antigen can result in a state of immune hyporesponsiveness or “tolerance.” The active mechanism appears to be a cytokine-mediated immune deviation away from a Th1 towards a predominantly Th2 and Th3 (i.e., TGF-β dominated) response. The active suppression with low dose antigen delivery can also suppress an unrelated immune response (bystander suppression) which is of considerable interest in the therapy of autoimmune diseases, for example, rheumatoid arthritis and SLE. Bystander suppression involves the secretion of Th1-counter-regulatory, suppressor cytokines in the local environment where proinflammatory and Th1 cytokines are released in either an antigen-specific or antigen-nonspecific manner. “Tolerance” as used herein is used to refer to this phenomenon. Indeed, oral tolerance has been effective in the treatment of a number of autoimmune diseases in animals including: experimental autoimmune encephalomyelitis (EAE), experimental autoimmune myasthenia gravis, collagen-induced arthritis (CIA), and insulin-dependent diabetes mellitus. In these models, the prevention and suppression of autoimmune disease is associated with a shift in antigen-specific humoral and cellular responses from a Th1 to Th2/Th3 response.

In another aspect, the present invention is directed to a kit including one or more of the compositions previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more nanoparticle cores, such as a nanoparticle core that comprises gold. A kit can also include one or more antagonists of nucleic acid-interacting complexes. A kit can also include one or more antigens.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

Four main potent sequences for inhibiting TLR9, ODN2088^{14,15}, ODN G^{12,17}, ODN MT01²², and ODN 4084F^{13,16} have been identified. These are shown in Table 1. We developed constructs using these sequences. These constructs are referred to in places herein as irSNAs. It is demonstrated herein that the irSNAs were efficacious in inhibiting TLR9 activation under a variety of stimulation conditions. To accomplish this task, we used the RAW-Blue murine macrophage reporter line system (Invivogen). Briefly, the RAW-Blue system consists of murine macrophages that stably express a reporter plasmid that is responsive to NFκB signaling downstream of TLR activation. This NFκB-controlled plasmid expresses an alkaline phosphatase that is secreted by the cell, can be collected, and quantified using a colorimetric detection agent to monitor TLR activation in live cells. We incubated RAW-Blue cells with increasing concentrations of either free irDNA oligonucleotides with either natural phosphodiester (po) or phosphorothioate (ps) backbones of each type or with irSNAs loaded with each respective immunoregulatory oligonucleotide (See Table 1) for two hours. After this incubation, the cells were stimulated with either 0.5 μM CpG-containing DNA with a phosphorothioate backbone or 10 μM CpG-containing DNA with a phosphodiester backbone, both known to stimulate TLR9^{1,10} and incubated the cells overnight. TLR9 activation was then measured using the reporter assay and relative activation levels plotted against concentration of immunoregulatory DNA or SNA constructs and IC₅₀ values determined using non-linear least squares regression fit assuming a Hill Slope of 1 using GraphPad Prism software.

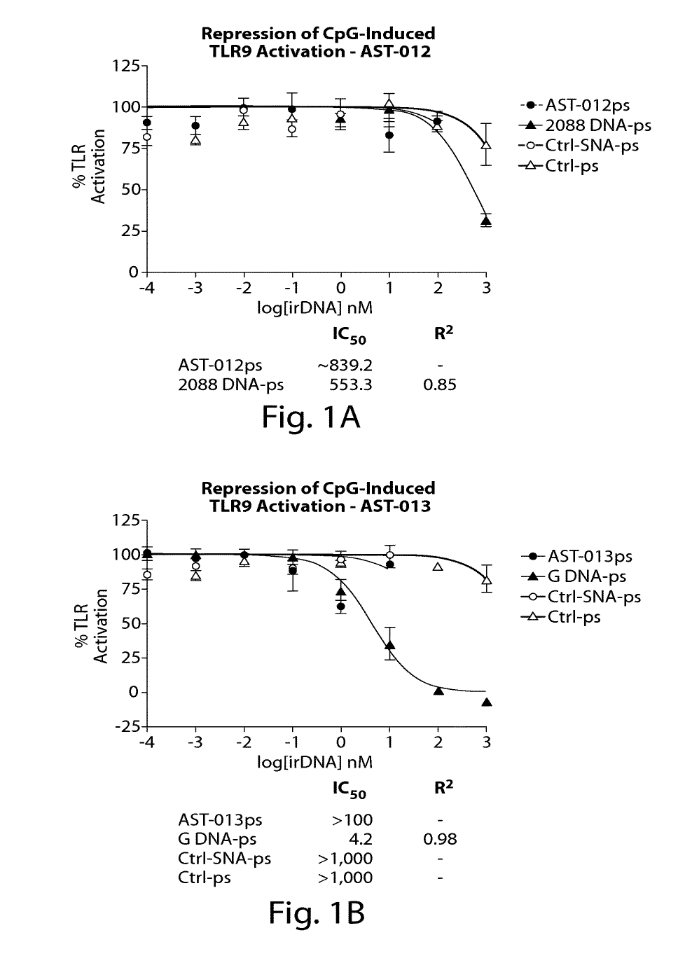

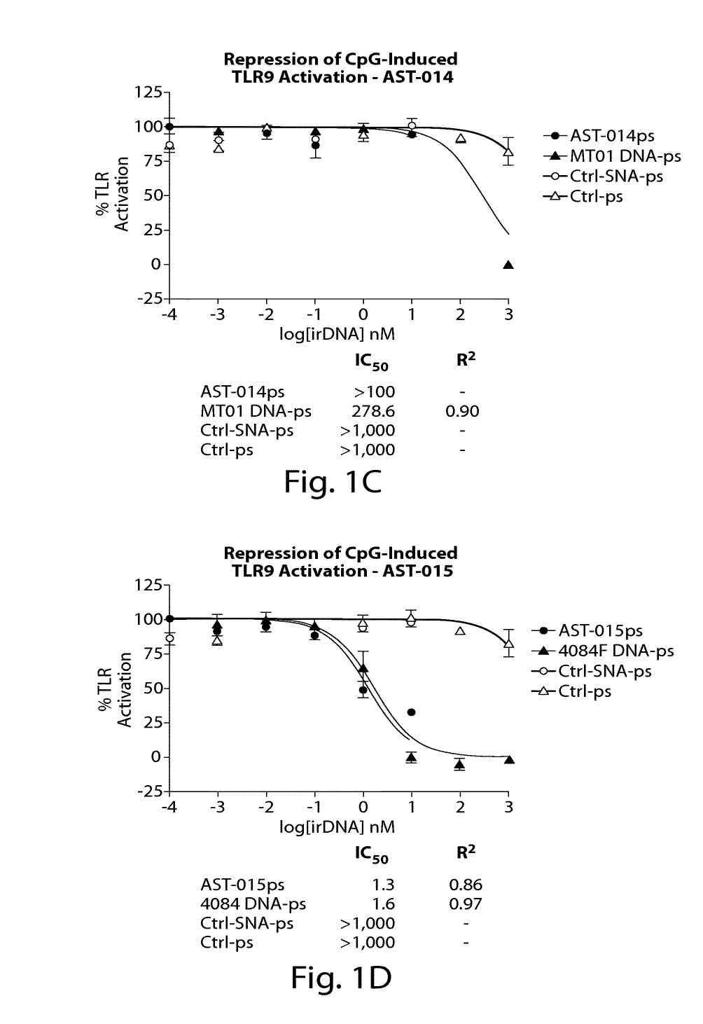

The results are shown in FIG. 1. 2088 DNA showed high nanomolar efficacy with an IC₅₀ of 553 nM while the corresponding irSNA construct, AST-012's, IC₅₀ values were unable to be determined due to dosing limits in the assay (FIG. 1A). G DNA showed a low nanomolar IC₅₀ of 4.2 nM, but was incompatible with stable incorporation into the SNA at high concentrations (FIG. 1B). MT01 DNA had an IC₅₀ of 278.6 nM and the IC50 of the respective irSNA, AST-014, was unable to be determined due to dosing limits in the assay (FIG. 1C). However, 4084F DNA showed the most potent efficacy with an IC₅₀ of 1.6 nM and the respective AST-015 SNA analog demonstrated equal efficacy with an IC₅₀ of 1.3 nM. This demonstrated that the AST-015 construct was as efficacious as free oligonucleotide and the irDNA sequence was compatible with the SNA architecture. Thus, as shown in FIG. 1 AST-015 was able to repress CpG-induced TLR9 activation in macrophage-like RAW-Blue cells.

### Example 2

Current therapies utilizing immunomodulatory oligonucleotides require the use of phosphorothioate backbones to prevent degradation of the oligonucleotide in biological fluids. However, phosphorothioate backbones introduce unwanted levels of toxicity and it is especially advantageous if natural phosphodiester backbones could be used. We tested the ability of SNAs incorporating natural phosphodiester chemistries to block TLR activity. We first examined if cells pre-incubated with the immunoregulatory constructs, either free 4084F DNA in po or ps chemistries, or AST-015 in po or ps chemistries, would cause macrophage-like cells (RAW-Blue) to become refractory to TLR9 antagonist CpG DNA. Briefly, the immunoregulatory constructs were added to cells in increasing concentrations for two hours to allow uptake and incorporation into endosomal compartments. Following this incubation, either 0.5 μM CpG DNA 1668ps or 10 μM CpG DNA 1826po were added and the cells incubated overnight and then stimulation was quantified as described above.

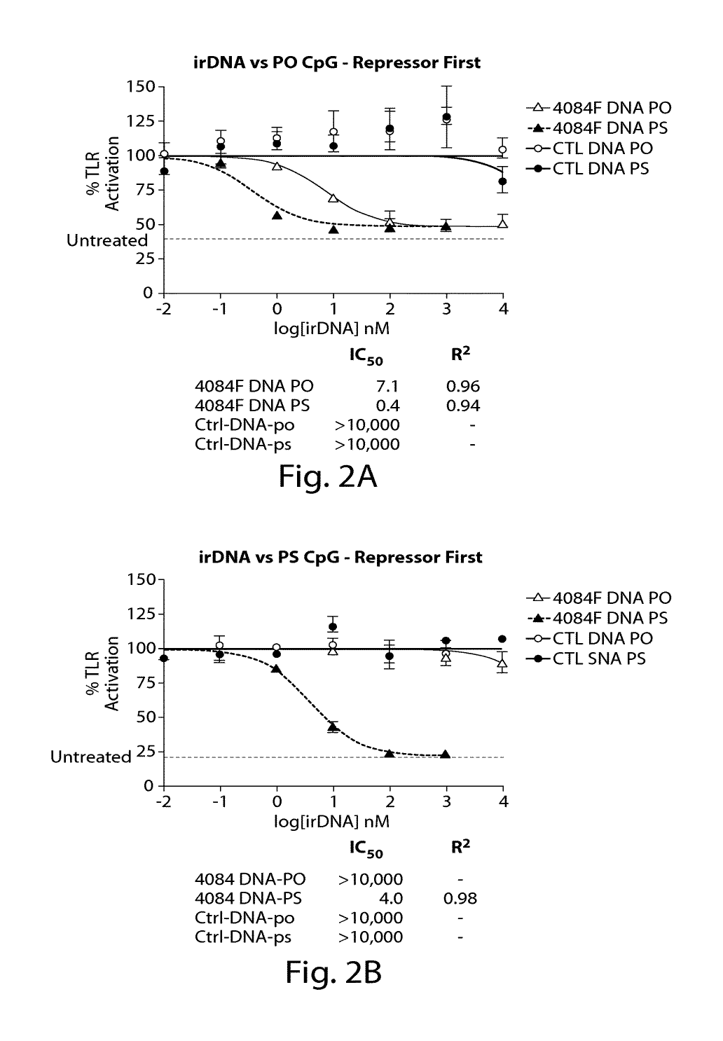

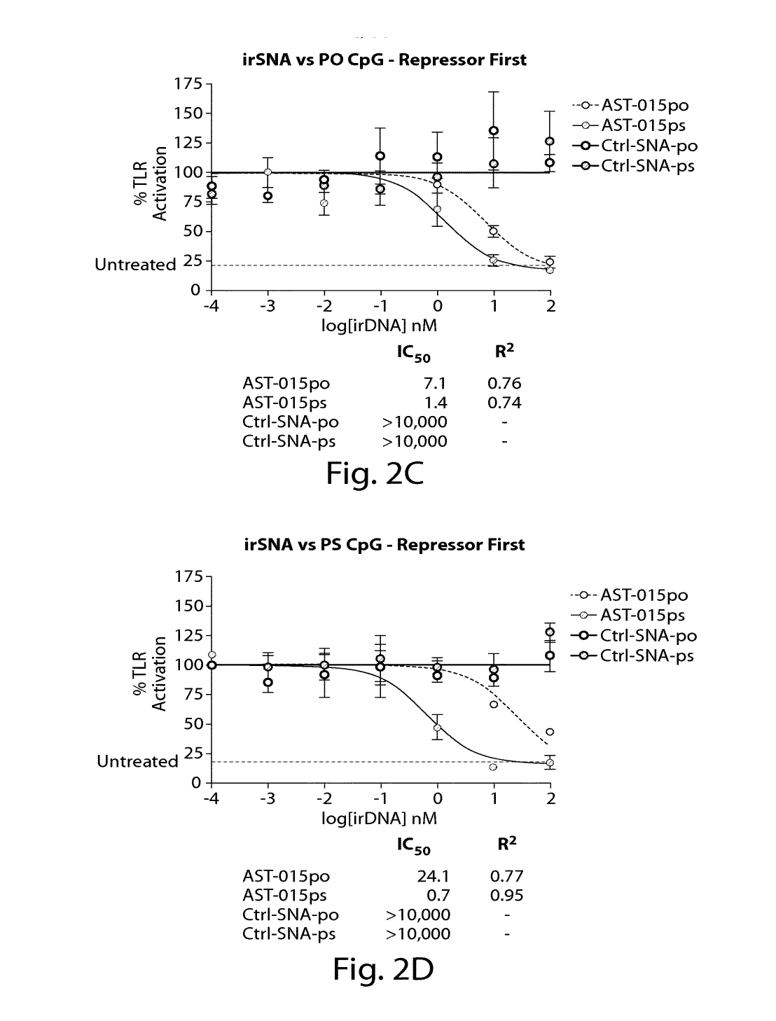

As a baseline for comparison, we examined the efficacy of the free 4084F DNA first. The results are shown in FIG. 2. When challenged with a phosphodiester CpG stimulatory DNA, 4084F DNApo had an IC₅₀ of 7.1 nM while the 4084F DNAps was about an order of magnitude more efficacious with an IC₅₀ of 0.4 nM. This demonstrates that both phosphodiester and phosphorothioate versions of free 4084F are capable of blocking free phosphodiester CpG-induced immuno stimulation (FIG. 2A). In the same system, when challenged with a more stable phosphorothioate CpG DNA, free 4084F DNApo was unable to block stimulation, however, 4084F DNAps was able to block stimulation with an IC₅₀ of 4 nM (FIG. 2B). With these values as a baseline for comparison we next determined the efficacy values for AST-015. When challenged with phosphodiester CpG DNA, AST-015po had an IC₅₀ of 7.1 nM, while AST015ps had an IC₅₀ of 1.4 nM, both nearly identical to that of free DNA (FIG. 2C). Interestingly, when challenged with the more stable phosphorothioate CpG DNA, AST-015po, whose free DNA analog previously was not efficacious against phosphorothioate CpG DNA, was efficacious with an IC₅₀ of 24.1 nM. AST-015ps treatment was also efficacious with an IC₅₀ of 0.7 nM (FIG. 2D). These data demonstrate that incorporation of immunoregulatory DNA sequences into the SNA architecture imparts unique and novel advantages over free DNA alone.

### Example 3

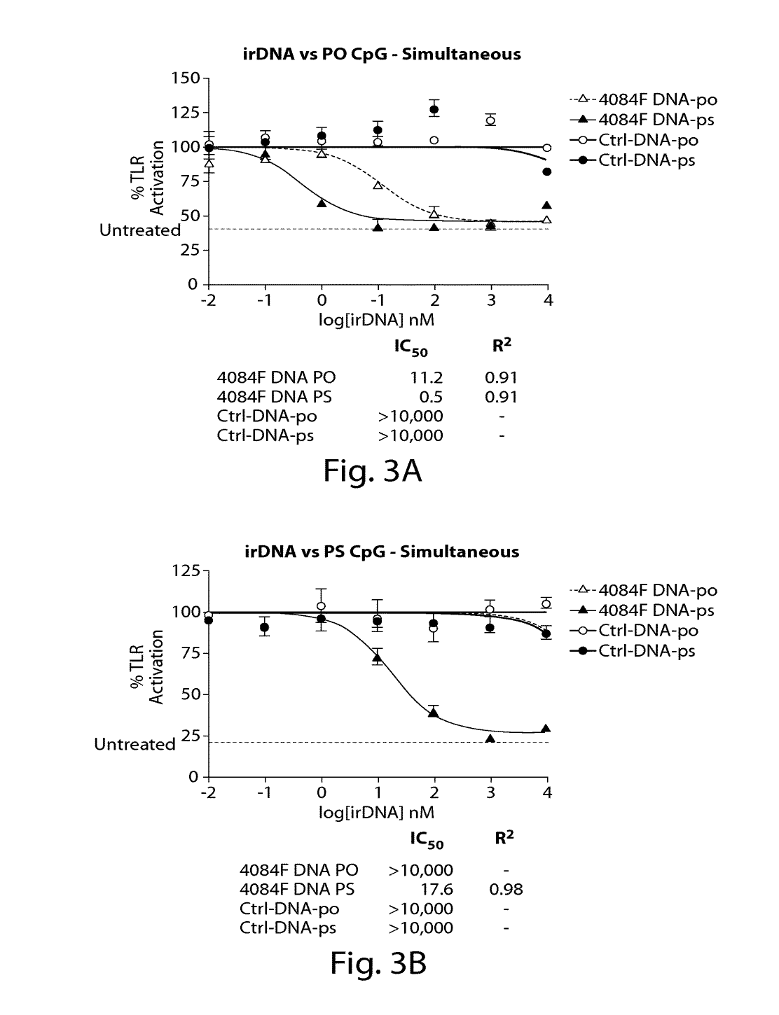

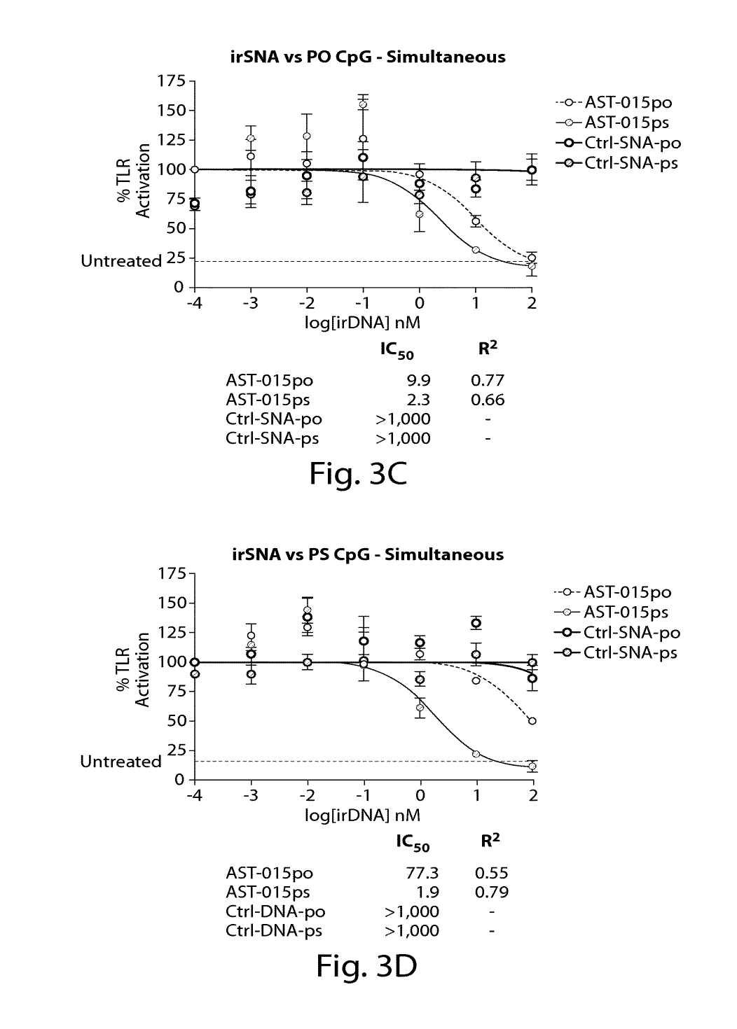

The previous Example examined the ability of AST-015 to repress TLR9-induced signaling when added prior to the stimulating CpG-containing immunostimulatory DNA. We next sought to determine if AST-015 would be able to out-compete the immunostimulatory DNA if added simultaneously. To accomplish this, RAW-Blue cells were stimulated with either 0.5 μM CpG DNA 1668ps or 10 μM CpG DNA 1826po along with increasing concentrations of either free 4084F DNA in both po and ps or AST-015 in both po and ps. The results are shown in FIG. 3.

When challenged simultaneously with a phosphodiester CpG stimulatory DNA, 4084F DNApo had an IC₅₀ of 11.2 nM while the 4084F DNAps was about an order of magnitude more efficacious with an IC₅₀ of 0.5 nM (FIG. 3A). In the same system, when challenged with a more stable phosphorothioate CpG DNA, free 4084F DNApo was unable to block stimulation, however, 4084F DNAps was able to block stimulation with an IC₅₀ of 17.6 nM (FIG. 3B). With these values as a baseline for comparison we next determined the efficacy values for AST-015. When challenged with phosphodiester CpG DNA, AST-015po had an IC₅₀ of 9.9 nM, while AST015ps had an IC₅₀ of 2.3 nM, both nearly identical to that of free DNA (FIG. 3C). Similar to the previously described pre-treatment with the irSNA, when challenged simultaneously with the more stable phosphorothioate CpG DNA, AST-015po, whose free DNA analog previously was not efficacious against phosphorothioate CpG DNA, was efficacious with an IC₅₀ of 77.3 nM. AST-015ps treatment was also efficacious with an IC₅₀ of 1.9 nM (FIG. 3D).

### Example 4

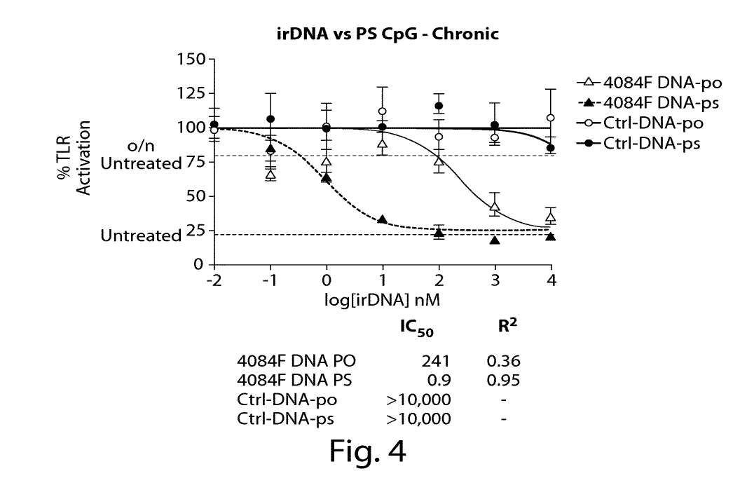

We next examined if the free 4084F DNA was able to repress CpG-induced TLR9 activation in cell that was already in a chronic stimulated state as a model for the clinical scenario where a patient is already presenting an over activated immune system. To accomplish this, RAW-Blue cells were stimulated with 0.5 μM CpG 1668ps for 18 hours to activate the macrophages and the media were replaced with an additional dose of 0.5 μM CpG1668ps along with increasing concentrations of free 4084F DNA in either po or ps chemistries for an additional 18 hours followed by quantification using the colorimetric detection assay. The results are shown in FIG. 4.

Free 4084F DNApo was efficacious in these chronically treated macrophages with an IC₅₀ of 241 nM. This is roughly two orders of magnitude less potent than the efficacy of 4084F DNAps which had an IC₅₀ of 0.9 nM suggesting that the free DNA in its phosphodiester form is a relatively poor repressor of TLR activation (FIG. 4).

Importantly these data demonstrate that pre-treatment of the immunoregulatory sequences is not required for the constructs to be efficacious. This is an important distinction as it enables the technology to be used both as a prophylactic and as an acute treatment when inflammatory symptoms present in the clinic.

Interestingly, AST-015 in po form shows low nanomolar efficacy against phosphorothioate TLR9-activating CpG-containing DNA, while its respective free DNA equivalent is ineffective in blocking this activation. AST-015 in ps form was equipotent against its free DNA equivalent. This demonstrates that immunoregulatory DNA can be loaded into the SNA construct without a loss of activity and with different efficacy than would be anticipated from a free DNA equivalent. In view of this data, oligonucleotide constructs can be designed with selective modification of the base sequences. For example selective incorporation of phosphorothioate backbones at specific sites and incorporation of lipophilic agents such as cholesterol, stearyl groups, and/or palmitoyl groups to promote membrane association can be made (See Table 2). This allows for the utilization of the advantageous properties of the SNA architecture such as enhanced degradation resistance in biological fluids, enhanced biodistribution, and rapid uptake of the construct in vivo to develop a more effective therapy compared to current free DNA treatment approaches.

### Example 5

### irSNAs can Antagonize Multiple TLRs

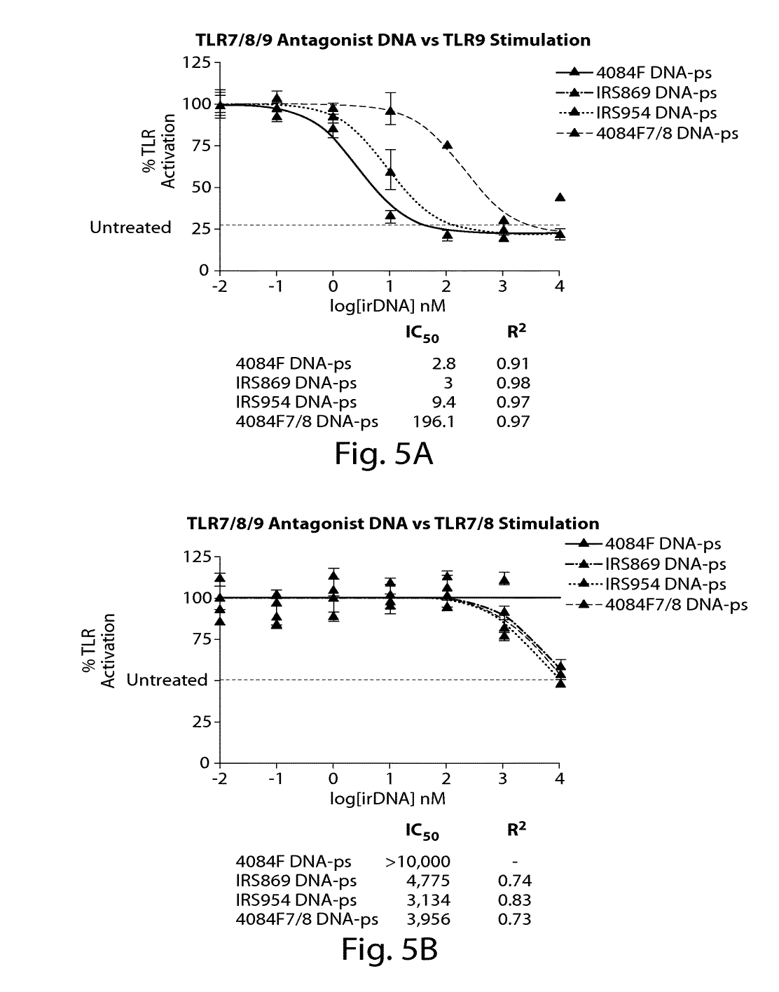

SNA constructs were tailored to incorporate customized regulatory sequences to serve as an antagonist to a broader range of TLRs. These constructs were compared against current state-of-the-art delivery of antagonists. To accomplish this novel TLR7/8/9 antagonist sequences were designed and compared for efficacy against current clinically relevant sequences developed by Dynavax^{22,24} (Table 3). Using the same system as described above, RAW-Blue cells were incubated with increasing concentrations of 4084F, IRS869, IRS954, or AST-developed 4084F7/8, all with phosphorothioate backbone chemistry, for two hours to allow for uptake and endosomal internalization. The cells were then challenged with either 0.5 μM of the TLR9 activating CpG 1668 DNA with phosphorothioate backbone, or with 5 μM of the TLR7/8 activating single stranded RNA, ssRNA 00 overnight and TLR activation was measured using the colorimetric assay described above. The results are shown in FIG. 5.

Interestingly the 4084F sequence that is incorporated into AST-015 was equally efficacious toward TLR9 as the state-of-the-art Dynavax sequences, IRS869 and IRS954 (IC₅₀s: 4084F=2.8 nM; IRS869=3.0 nM; IRS954=9.4 nM) and the AST-developed TLR7/8/9 antagonist 4084F7/8 had a weaker but still efficacious IC₅₀ of 196 nM (FIG. 5A). Importantly, 4084F7/8 gained the ability to antagonize TLR7/8 activation versus its base sequence counterpart 4084F with the same efficacy as the Dynavax sequences (IC₅₀s: 4084F>10,000 nM; IRS869=4,775 nM; IRS954=3,134 nM; 4084F7/8=3,956 nM) (FIG. 5B).

Importantly, this example demonstrates that specific sequences can be designed to antagonize a range of endosomal TLRs through modification of the base sequence. Based on these data, the skilled artisan can develop both specific TLR antagonists and broad TLR antagonists in SNA form that will either perform equally to or better than their free DNA counterparts or current state-of-the-art clinically tested constructs.

### Example 6

### Liposomal Spherical Nucleic Acid Antagonism of Various Toll-Like Receptor Agonist Activity

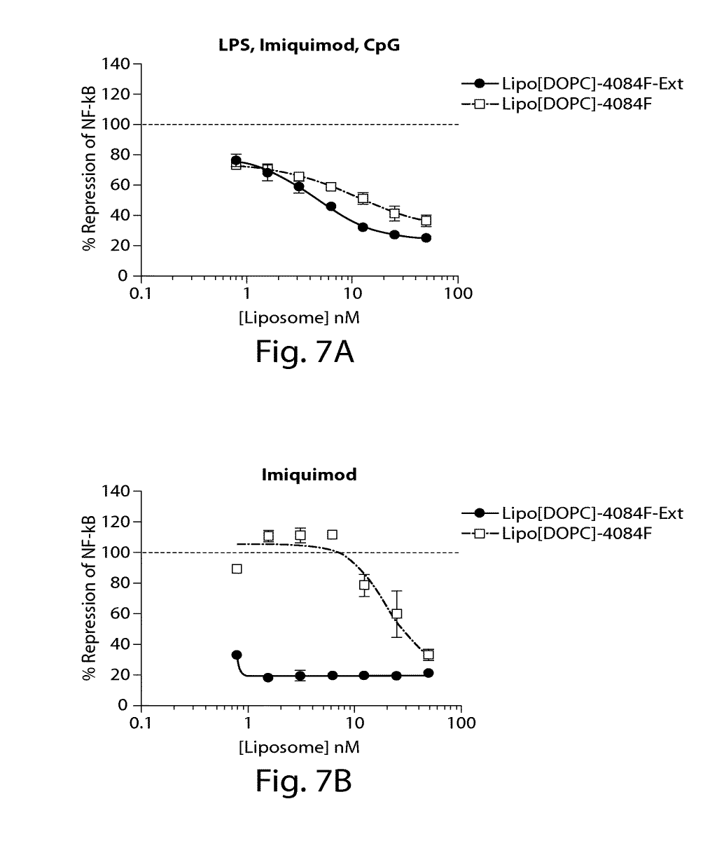

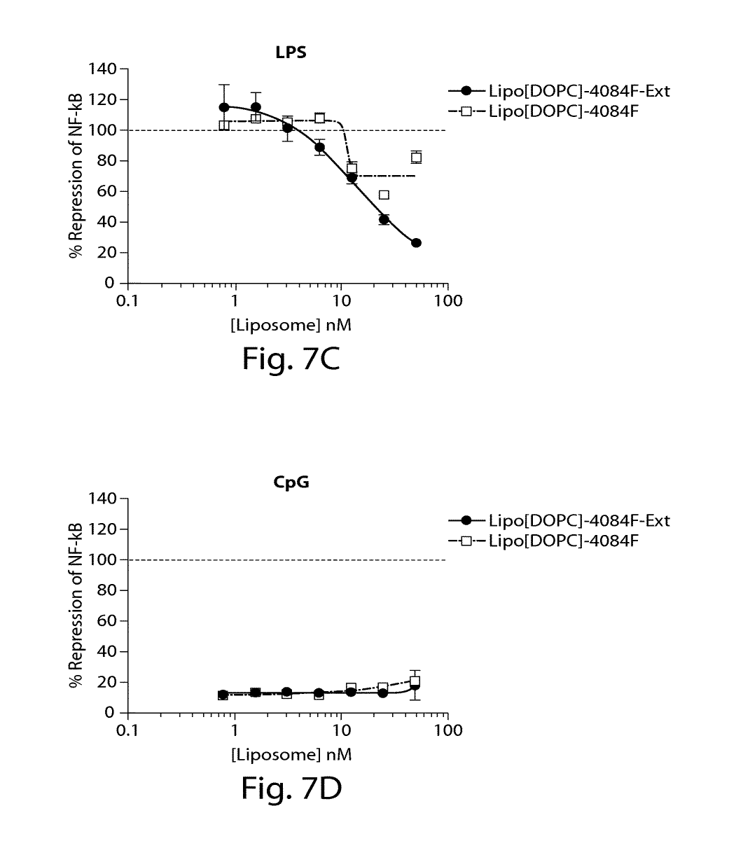

Liposomal SNAs were prepared by forming a lipid micelle core consisting of DOPC (1,2-dioleoyl-sn-glycero-3-phosphocholine) formed by a conventional liposome extrusion process. Following DOPC micelle formation, oligonucleotides of sequence 4084F (5′-C*C*T*G*G*A*T*G*G*G*A*A-3′ (SEQ ID NO: 121), *indicates phosphorothioate) or 4084F-Ext (5′-T*G*C*T*T*G*A*C*A*C*C*T*G*G*A*T*G*G*G*A*A-3′)(SEQ ID NO: 122) were attached to a lipid group at the 3′ end, such as distearyl or tocopherol and incorporated into the micelle through simple mixing, followed by purification by tangential flow filtration (TFF) to achieve purified liposomal SNAs with approximately ˜100 oligos/SNA. RAW-Blue Macrophages (InVivoGen) were incubated with the indicated TLR agonist, either imiquimod (TLR7, FIG. 7B), CpG 1826 (CpG, TLR9, FIG. 7D), bacterial lipopolysaccharide (LPS, TLR4, FIG. 7C), or all three simultaneously (FIG. 7A) for 4 hours followed by overnight incubation with the indicated liposomal SNA or PBS and referenced to untreated. The data is shown in FIG. 7. It was found that both constructs are able to block stimulation by all three agonists.

TABLE 1
Identity of TLR9 antagonists and stimulatory sequences used in this study.
All sequences consist of deoxyribonucleotides. /iSp18/ = internal Spacer 18 linker;
/3ThioMC3-D/ = 3′ terminal thiol with 3 carbon linker; 13 nm AuNP = 13 nanometer diameter
gold nanoparticle; ″po″ in text refers to all phosphodiester backbone chemistry; ″ps″
in text refers to all phosphorothioate backbone chemistry

| | | | |
|---|---|---|---|
| Name | Sequence | Ref. | SEQ ID NO: |

Immunoregulatory Sequences

| | | | |
|---|---|---|---|
| 2088 | TTCTGGCGGGGAAGT/iSp18//iSp18//3ThioMC3-D/14,2591 | 14,25 | 91 |
| DNA | | | |
| G | CTCCTATTGGGGGTTTCCTAT/iSp18//iSp18//3ThioMC3-D/ | 12,17 | 92 |
| DNA | | | |
| MT01 | ACCCCCTCTACCCCCTCTACCCCTCT/iSp18//iSp18//3ThioMC3-D/ | 22 | 93 |
| DNA | | | |
| 4084F | CCTGGATGGGAA/iSp18//iSp18//3ThioMC3-D/ | 13,16 | 94 |
| DNA | | | |
| AST- | TTCTGGCGGGGAAGT/iSp18//iSp18//3ThioMC3-D/(13 nm AuNP) | AST | 95 |
| 012 | | | |
| AST- | CTCCTATTGGGGGTTTCCTAT/iSp18//iSp18//3ThioMC3-D/(13 nm | AST | 96 |
| 013 | AuNP) | | |
| AST- | ACCCCCTCTACCCCCTCTACCCCTCT/iSp18//iSp18//3ThioMC3-D/ | AST | 97 |
| 014 | (13 nm AuNP) | | |
| AST- | CCTGGATGGGAA/iSp18//iSp18//3ThioMC3-D/(13 nm AuNP) | AST | 98 |
| 015 | | | |
| Ctrl | TCCTGAGCTTGAAGT/iSp18//iSp18//3ThioMC3-D/ | 14,25 | 99 |
| DNA | | | |
| Ctrl | TCCTGAGCTTGAAGT/iSp18//iSp18//3ThioMC3-D/(13 nm AuNP) | AST | 100 |
| SNA | | | |

Immunostimulatory Sequences

| | | | |
|---|---|---|---|
| CpG | TCCATGACGTTCCTGACGTT | 5,10 | 101 |
| 1826 | | | |
| CpG | TCCATGACGTTCCTGATGCT | 5,12 | 102 |
| 1668 | | | |

TABLE 2
Conceived modifications to AST-015 to promote efficacy
All sequences consist of deoxyribonucleotides. /iSp18/ = internal Spacer 18
linker; /3ThioMC3-D/ = 3′ terminal thiol with 3 carbon linker; 13 nm
AuNP = 13 nanometer diameter gold nanoparticle; * = phosphorothioate
linkage; /Chol/ = Cholesterol; /Stryl/ = C16/C18 Stearyl group;
/Palm/ = Palmitoyl group.
Immunoregulatory Sequences

| Name | Sequence | Ref. | SEQ ID NO: |
|---|---|---|---|
| AST- | C*C*T*GGATGGGAA/iSp18//iSp18//3ThioMC3-D/(13 nm | AST | 103 |
| 015mod1 | AuNP) | | |
| AST- | CCTGGATG*G*G*AA/iSp18//iSp18//3ThioMC3-D/(13 nm | AST | 104 |
| 015mod2 | AuNP) | | |
| AST- | C*C*T*GGATG*G*G*AA/iSp18//iSp18//3ThioMC3-D/(13 | AST | 105 |
| 015mod3 | nm AuNP) | | |
| AST- | /Chol/CCTGGATGGGAA/iSp18//iSp18//3ThioMC3-D/(13 | AST | 106 |
| 015mod4 | nm AuNP) | | |
| AST- | /Stryl/CCTGGATGGGAA/iSp18//iSp18//3ThioMC3-D/(13 | AST | 107 |
| 015mod5 | nm AuNP) | | |
| AST- | /Palm/CCTGGATGGGAA/iSp18//iSp18//3ThioMC3-D/(13 | AST | 108 |
| 015mod6 | nm AuNP) | | |

TABLE 3
Identity TLR7/8/9 antagonists and
stimulatory sequences used in this study.

| | | | |
|---|---|---|---|
| | | | SEQ |
| Name | Sequence | Ref. | ID NO: |

Immunoregulatory Sequences

| | | | |
|---|---|---|---|
| IRS869 | TCCTGGAGGGGTTGT | 24 | 109 |
| IRS954 | TGCTCCTGGAGGGGTTGT | 24 | 110 |
| 4084F7/8DNA | TGCTGGATGGGAA | AST | 111 |
| 4084F7/8Ext | TGCCCTGGATGGGAA | AST | 112 |
| 4084D7/8Full | TGCTTGACACCTGGATGGGAA | AST | 113 |
| AST-016 | TGCTGGATGGGAA/iSp18//iSp18// | AST | 114 |
| | 3ThioMC3-D/(13 nm AuNP) | | |
| AST-017 | TGCCCTGGATGGGAA/iSp18//iSp18// | AST | 115 |
| | 3ThioMC3-D/(13 nm AuNP) | | |
| AST-018 | TGCTTGACACCTGGATGGGAA/iSp18// | AST | 116 |
| | iSp18//3ThioMC3-D/(13 nm AuNP) | | |
| Ctrl DNA | TCCTGAGCTTGAAGT/iSp18//iSp18// | 24 | 117 |
| | 3ThioMC3-D/ | | |
| Ctrl SNA | TCCTGAGCTTGAAGT/iSp18//iSp18// | AST | 118 |
| | 3ThioMC3-D/(13 nm AuNP) | | |

Immunostimulatory Sequences

| | | | |
|---|---|---|---|
| ssRNA06 | TCCATGACGTTCCTGACGTT | 6-8 | 119 |
| CpG 1668 | TCCATGACGTTCCTGATGCT | 10 | 120 |

All sequences consist of deoxyribonucleotides, except ssRNA06 which consists of ribonucleotides.
/iSp18/ = internal Spacer 18 linker;
/3ThioMC3-D/ = 3′ terminal thiol with 3 carbon linker;
13 nm AuNP = 13 nanometer diameter gold nanoparticle;
“po” in text refers to all phosphodiester backbone chemistry;
“ps” in text refers to all phosphorothioate backbone chemistry.

### REFERENCES

(1) Hornung, V.; Rothenfusser, S.; Britsch, S.; Krug, A.; Jahrsdorfer, B.; Giese, T.; Endres, S.; Hartmann, G. J Immunol 2002, 168, 4531.
(2) Stacey, K. J.; Sweet, M. J.; Hume, D. A. J Immunol 1996, 157, 2116.
(3) Zarember, K. A.; Godowski, P. J. J Immunol 2002, 168, 554.
(4) Bauer, M.; Redecke, V.; Ellwart, J. W.; Scherer, B.; Kremer, J. P.; Wagner, H.; Lipford, G. B. J Immunol 2001, 166, 5000.
(5) Bauer, S.; Kirschning, C. J.; Hacker, H.; Redecke, V.; Hausmann, S.; Akira, S.; Wagner, H.; Lipford, G. B. Proceedings of the National Academy of Sciences of the United States of America 2001, 98, 9237.
(6) Diebold, S. S.; Massacrier, C.; Akira, S.; Paturel, C.; Morel, Y.; Reis e Sousa, C. European journal of immunology 2006, 36, 3256.
(7) Forsbach, A.; Nemorin, J. G.; Montino, C.; Muller, C.; Samulowitz, U.; Vicari, A. P.; Jurk, M.; Mutwiri, G. K.; Krieg, A. M.; Lipford, G. B.; Vollmer, J. J Immunol 2008, 180, 3729.
(8) Heil, F.; Hemmi, H.; Hochrein, H.; Ampenberger, F.; Kirschning, C.; Akira, S.; Lipford, G.; Wagner, H.; Bauer, S. Science 2004, 303, 1526.
(9) Krieg, A. M. Journal of clinical immunology 1995, 15, 284.
(10) Krieg, A. M.; Yi, A. K.; Matson, S.; Waldschmidt, T. J.; Bishop, G. A.; Teasdale, R.; Koretzky, G. A.; Klinman, D. M. Nature 1995, 374, 546.
(11) West, A. P.; Koblansky, A. A.; Ghosh, S. Annual review of cell and developmental biology 2006, 22, 409.
(12) Peter, M.; Bode, K.; Lipford, G. B.; Eberle, F.; Heeg, K.; Dalpke, A. H. Immunology 2008, 123, 118.
(13) Lenert, P.; Yi, A. K.; Krieg, A. M.; Stunz, L. L.; Ashman, R. F. Antisense & nucleic acid drug development 2003, 13, 143.
(14) Stunz, L. L.; Lenert, P.; Peckham, D.; Yi, A. K.; Haxhinasto, S.; Chang, M.; Krieg, A. M.; Ashman, R. F. European journal of immunology 2002, 32, 1212.
(15) Krieg, J.; Hartmann, S.; Vicentini, A.; Glasner, W.; Hess, D.; Hofsteenge, J. Molecular biology of the cell 1998, 9, 301.
(16) Lenert, P.; Rasmussen, W.; Ashman, R. F.; Ballas, Z. K. DNA and cell biology 2003, 22, 621.
(17) Gursel, I.; Gursel, M.; Yamada, H.; Ishii, K. J.; Takeshita, F.; Klinman, D. M. J Immunol 2003, 171, 1393.
(18) Goh, F. G.; Midwood, K. S. Rheumatology (Oxford) 2012, 51, 7.
(19) Chabaud, M.; Durand, J. M.; Buchs, N.; Fossiez, F.; Page, G.; Frappart, L.; Miossec, P. Arthritis and rheumatism 1999, 42, 963.
(20) van den Berg, W. B.; Miossec, P. Nature reviews. Rheumatology 2009, 5, 549.
(21) Emery, P.; Fleischmann, R.; Filipowicz-Sosnowska, A.; Schechtman, J.; Szczepanski, L.; Kavanaugh, A.; Racewicz, A. J.; van Vollenhoven, R. F.; Li, N. F.; Agarwal, S.; Hessey, E. W.; Shaw, T. M. Arthritis and rheumatism 2006, 54, 1390.
(22) Yang, G.; Wan, M.; Zhang, Y.; Sun, L.; Sun, R.; Hu, D.; Zhou, X.; Wang, L.; Wu, X.; Yu, Y. Immunology 2010, 131, 501.
(23) Kanzler, H.; Barrat, F. J.; Hessel, E. M.; Coffman, R. L. Nature medicine 2007, 13, 552.
(24) Barrat, F. J.; Meeker, T.; Gregorio, J.; Chan, J. H.; Uematsu, S.; Akira, S.; Chang, B.; Duramad, O.; Coffman, R. L. The Journal of experimental medicine 2005, 202, 1131.
(25) Krieg, A. M.; Wu, T.; Weeratna, R.; Efler, S. M.; Love-Homan, L.; Yang, L.; Yi, A. K.; Short, D.; Davis, H. L. Proceedings of the National Academy of Sciences of the United States of America 1998, 95, 12631.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A nanoscale construct comprising:
a corona of an antagonist of nucleic acid-interacting complexes wherein the surface density of the antagonist of nucleic acid-interacting complexes is at least 0.3 pmol/cm2.

2. A nanoscale construct comprising:
a corona of an antagonist of nucleic acid-interacting complexes, and an antigen incorporated into the corona, wherein the surface density of the antigen is at least 0.3 pmol/cm2.

3. The nanoscale construct of claim 2, wherein the antigen includes at least two different types of antigen.

4. A nanoscale construct comprising:
a corona with at least two antagonists of nucleic acid-interacting complexes incorporated, wherein the antagonists are selected from the group consisting of TLR 3, 7/8, and/or 9 antagonists.

5. The nanoscale construct of any one of claims 1-4, wherein the antagonist of nucleic acid-interacting complexes contains a spacer.

6. The nanoscale construct of any one of claims 1-5, wherein the antagonist of nucleic acid-interacting complexes is RNA or DNA.

7. The nanoscale construct of claim 6, wherein the antagonists of nucleic acid-interacting complexes is a double stranded RNA or double stranded DNA.

8. The nanoscale construct of claim 6, wherein the antagonist of nucleic acid-interacting complexes is a single stranded RNA.

9. The nanoscale construct of any one of claims 1-8, wherein the surface density of the antagonist of nucleic acid-interacting complexes is at least 15 pmol/cm².

10. The nanoscale construct of any one of claims 1-8, wherein the surface density of the antagonist of nucleic acid-interacting complexes is at least 45 pmol/cm².

11. The nanoscale construct of claim 6, wherein the antagonist of nucleic acid-interacting complexes is an unmethylated deoxyribonucleic acid.

12. The nanoscale construct of claim 11, wherein the unmethylated deoxyribonucleic acid contains an optimized immunoregulatory sequence.

13. The nanoscale construct of any one of claims 1-12, wherein the nanoscale construct contains a nanoparticle core which is metallic.

14. The nanoscale construct of claim 13, wherein the metal core is selected from the group consisting of gold, silver, platinum, aluminum, palladium, copper, cobalt, indium, nickel and mixtures thereof.

15. The nanoscale construct of claim 13, wherein the nanoparticle core comprises gold.

16. The nanoparticle construct of any one of claims 1-16, wherein the nanoscale construct is degradable.

17. The nanoscale construct of any one of claims 1-16, wherein the diameter of the nanoscale construct is from 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 ran in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter.

18. A nanoscale construct comprising;
a spherical corona of an antagonist of nucleic acid-interacting complexes, wherein the antagonist is nucleic acid having at least one phosphodiester internucleotide linkage.

19. The nanoscale construct of claim 18, wherein the antagonist is a CpG oligonucleotide.

20. The nanoscale construct of any one of claims 18-19, wherein each internucleotide linkage of the nucleic acid is a phosphodiester linkage.

21. The nanoscale construct of any one of claims 1-20, wherein the corona is a spherical corona.

22. A vaccine comprising a nanoscale construct of any of claims 1-21 and a carrier.

23. A method for delivering a therapeutic agent to a cell comprising delivering the nanoscale construct of any one of claims 1-21 to the cell.

24. A method for regulating expression of a target molecule comprising delivering the nanoscale construct of any one of claims 1-21 to the cell.

25. The method of claim 24, wherein the target molecule is a TLR selected from the group consisting of TLR3, 7, 8, and 9.

26. A method for antagonizing a TLR comprising delivering the nanoscale construct of any one of claims 1-21 to the cell.

27. A method of treating a subject, comprising
administering to the subject the nanoscale construct of any one of claims 1-21 in an effective amount to reduce an immune response.

28. The method of claim 27, wherein the subject has an infectious disease.

29. The method of claim 27, wherein the subject has an inflammation induced cancer.

30. The method of claim 27, wherein the subject has an autoimmune disease.

31. The method of claim 27, wherein the subject has an allergy.

32. The method of claim 27, wherein the subject has an allergic disease.

33. The method of claim 27, wherein the subject has an inflammatory disease.

34. The method of claim 27, wherein the subject has a metabolic disease.

35. The method of claim 27 wherein the subject has a cardiovascular disease.

36. The method of claim 27 wherein the subject is a candidate for or the recipient of tissue or organ transplant.

37. A method of modulating an immune response in a subject, comprising administering to the subject the nanoscale construct of any one of claims 18-21 in an effective amount to modulate an immune response.


SEQUENCE LISTING (120 sequences)

SEQ ID NO: 1 - DNA, 15 bp, Artificial Sequence
  Synthetic Polynucleotide
tcctggaggggttgt

SEQ ID NO: 2 - DNA, 18 bp, Artificial Sequence
  Synthetic Polynucleotide
tgctcctggaggggttgt

SEQ ID NO: 3 - DNA, 13 bp, Artificial Sequence
  Synthetic Polynucleotide
tgctggatgggaa

SEQ ID NO: 4 - DNA, 15 bp, Artificial Sequence
  Synthetic Polynucleotide
tgccctggatgggaa

SEQ ID NO: 5 - DNA, 21 bp, Artificial Sequence
  Synthetic Polynucleotide
tgcttgacacctggatgggaa

SEQ ID NO: 6 - DNA, 13 bp, Artificial Sequence
  Synthetic Polynucleotide
tgctggatgggaa

SEQ ID NO: 7 - DNA, 15 bp, Artificial Sequence
  Synthetic Polynucleotide
tgccctggatgggaa

SEQ ID NO: 8 - DNA, 21 bp, Artificial Sequence
  Synthetic Polynucleotide
tgcttgacacctggatgggaa

SEQ ID NO: 9 - DNA, 15 bp, Artificial Sequence
  Synthetic Polynucleotide
tcctgagcttgaagt

SEQ ID NO: 10 - DNA, 15 bp, Artificial Sequence
  Synthetic Polynucleotide
tcctgagcttgaagt

SEQ ID NO: 11 - DNA, 15 bp, Artificial Sequence
  Synthetic Polynucleotide
ttctggcggggaagt

SEQ ID NO: 12 - DNA, 21 bp, Artificial Sequence
  Synthetic Polynucleotide
ctcctattgggggtttcctat

SEQ ID NO: 13 - DNA, 26 bp, Artificial Sequence
  Synthetic Polynucleotide
accccctctaccccctctacccctct

SEQ ID NO: 14 - DNA, 12 bp, Artificial Sequence
  Synthetic Polynucleotide
cctggatgggaa

SEQ ID NO: 15 - DNA, 15 bp, Artificial Sequence
  Synthetic Polynucleotide
ttctggcggggaagt

SEQ ID NO: 16 - DNA, 21 bp, Artificial Sequence
  Synthetic Polynucleotide
ctcctattgggggtttcctat

SEQ ID NO: 17 - DNA, 26 bp, Artificial Sequence
  Synthetic Polynucleotide
accccctctaccccctctacccctct

SEQ ID NO: 18 - DNA, 12 bp, Artificial Sequence
  Synthetic Polynucleotide
cctggatgggaa

SEQ ID NO: 19 - DNA, 12 bp, Artificial Sequence
  Synthetic Polynucleotide
cctggatgggaa

SEQ ID NO: 20 - DNA, 12 bp, Artificial Sequence
  Synthetic Polynucleotide
cctggatgggaa

SEQ ID NO: 21 - DNA, 12 bp, Artificial Sequence
  Synthetic Polynucleotide
cctggatgggaa

SEQ ID NO: 22 - DNA, 12 bp, Artificial Sequence
  Synthetic Polynucleotide
cctggatgggaa

SEQ ID NO: 23 - DNA, 12 bp, Artificial Sequence
  Synthetic Polynucleotide
cctggatgggaa

SEQ ID NO: 24 - DNA, 12 bp, Artificial Sequence
  Synthetic Polynucleotide
cctggatgggaa

SEQ ID NO: 25 - DNA, 15 bp, Artificial Sequence
  Synthetic Polynucleotide
tcctggaggggttgt

SEQ ID NO: 26 - DNA, 18 bp, Artificial Sequence
  Synthetic Polynucleotide
tgctcctggaggggttgt

SEQ ID NO: 27 - DNA, 13 bp, Artificial Sequence
  Synthetic Polynucleotide
tgctggatgggaa

SEQ ID NO: 28 - DNA, 15 bp, Artificial Sequence
  Synthetic Polynucleotide
tgccctggatgggaa

SEQ ID NO: 29 - DNA, 21 bp, Artificial Sequence
  Synthetic Polynucleotide
tgcttgacacctggatgggaa

SEQ ID NO: 30 - DNA, 13 bp, Artificial Sequence
  Synthetic Polynucleotide
tgctggatgggaa

SEQ ID NO: 31 - DNA, 15 bp, Artificial Sequence
  Synthetic Polynucleotide
tgccctggatgggaa

SEQ ID NO: 32 - DNA, 21 bp, Artificial Sequence
  Synthetic Polynucleotide
tgcttgacacctggatgggaa

SEQ ID NO: 33 - DNA, 15 bp, Artificial Sequence
  Synthetic Polynucleotide
tcctgagcttgaagt

SEQ ID NO: 34 - DNA, 15 bp, Artificial Sequence
  Synthetic Polynucleotide
tcctgagcttgaagt

SEQ ID NO: 35 - DNA, 15 bp, Artificial Sequence
  Synthetic Polynucleotide
ttctggcggggaagt

SEQ ID NO: 36 - DNA, 21 bp, Artificial Sequence
  Synthetic Polynucleotide
ctcctattgggggtttcctat

SEQ ID NO: 37 - DNA, 26 bp, Artificial Sequence
  Synthetic Polynucleotide
accccctctaccccctctacccctct

SEQ ID NO: 38 - DNA, 12 bp, Artificial Sequence
  Synthetic Polynucleotide
cctggatgggaa

SEQ ID NO: 39 - DNA, 15 bp, Artificial Sequence
  Synthetic Polynucleotide
ttctggcggggaagt

SEQ ID NO: 40 - DNA, 21 bp, Artificial Sequence
  Synthetic Polynucleotide
ctcctattgggggtttcctat

SEQ ID NO: 41 - DNA, 26 bp, Artificial Sequence
  Synthetic Polynucleotide
accccctctaccccctctacccctct

SEQ ID NO: 42 - DNA, 12 bp, Artificial Sequence
  Synthetic Polynucleotide
cctggatgggaa

SEQ ID NO: 43 - DNA, 13 bp, Artificial Sequence
  Synthetic Polynucleotide
tgctggatgggaa

SEQ ID NO: 44 - DNA, 15 bp, Artificial Sequence
  Synthetic Polynucleotide
tgccctggatgggaa

SEQ ID NO: 45 - DNA, 21 bp, Artificial Sequence
  Synthetic Polynucleotide
tgcttgacacctggatgggaa

SEQ ID NO: 46 - DNA, 15 bp, Artificial Sequence
  Synthetic Polynucleotide
tcctgagcttgaagt

SEQ ID NO: 47 - DNA, 15 bp, Artificial Sequence
  Synthetic Polynucleotide
tcctgagcttgaagt

SEQ ID NO: 48 - DNA, 14 bp, Artificial Sequence
  Synthetic Polynucleotide
tctggcggggaagt

SEQ ID NO: 49 - DNA, 21 bp, Artificial Sequence
  Synthetic Polynucleotide
ctcctattgggggtttcctat

SEQ ID NO: 50 - DNA, 26 bp, Artificial Sequence
  Synthetic Polynucleotide
accccctctaccccctctacccctct

SEQ ID NO: 51 - DNA, 12 bp, Artificial Sequence
  Synthetic Polynucleotide
cctggatgggaa

SEQ ID NO: 52 - DNA, 15 bp, Artificial Sequence
  Synthetic Polynucleotide
ttctggcggggaagt

SEQ ID NO: 53 - DNA, 21 bp, Artificial Sequence
  Synthetic Polynucleotide
ctcctattgggggtttcctat

SEQ ID NO: 54 - DNA, 26 bp, Artificial Sequence
  Synthetic Polynucleotide
accccctctaccccctctacccctct

SEQ ID NO: 55 - DNA, 12 bp, Artificial Sequence
  Synthetic Polynucleotide
cctggatgggaa

SEQ ID NO: 56 - DNA, 24 bp, Artificial Sequence
  Synthetic Polynucleotide
ttagggttagggttagggttaggg

SEQ ID NO: 57 - DNA, 24 bp, Artificial Sequence
  Synthetic Polynucleotide
ttagggttagggttagggttaggg

SEQ ID NO: 58 - DNA, 24 bp, Artificial Sequence
  Synthetic Polynucleotide
ttagggttagggttagggttaggg

SEQ ID NO: 59 - DNA, 24 bp, Artificial Sequence
  Synthetic Polynucleotide
ttagggttagggttagggttaggg

SEQ ID NO: 60 - DNA, 24 bp, Artificial Sequence
  Synthetic Polynucleotide
ttagggttagggttagggttaggg

SEQ ID NO: 61 - DNA, 24 bp, Artificial Sequence
  Synthetic Polynucleotide
ttagggttagggttagggttaggg

SEQ ID NO: 62 - DNA, 16 bp, Artificial Sequence
  Synthetic Polynucleotide
ctatctgcgttctctg

SEQ ID NO: 63 - DNA, 16 bp, Artificial Sequence
  Synthetic Polynucleotide
ctatctgcgttctctg

SEQ ID NO: 64 - DNA, 16 bp, Artificial Sequence
  Synthetic Polynucleotide
ctatctgcgttctctg

SEQ ID NO: 65 - DNA, 16 bp, Artificial Sequence
  Synthetic Polynucleotide
ctatctgcgttctctg

SEQ ID NO: 66 - DNA, 16 bp, Artificial Sequence
  Synthetic Polynucleotide
ctatctgcgttctctg

SEQ ID NO: 67 - DNA, 16 bp, Artificial Sequence
  Synthetic Polynucleotide
ctatctgcgttctctg

SEQ ID NO: 68 - DNA, 24 bp, Artificial Sequence
  Synthetic Polynucleotide
ttagggttagggttagggttaggg

SEQ ID NO: 69 - DNA, 24 bp, Artificial Sequence
  Synthetic Polynucleotide
ttagggttagggttagggttaggg

SEQ ID NO: 70 - DNA, 16 bp, Artificial Sequence
  Synthetic Polynucleotide
ctatctgcgttctctg

SEQ ID NO: 71 - DNA, 16 bp, Artificial Sequence
  Synthetic Polynucleotide
ctatctgcgttctctg

SEQ ID NO: 72 - DNA, 13 bp, Artificial Sequence
  Synthetic Polynucleotide
tgctggatgggaa

SEQ ID NO: 73 - DNA, 15 bp, Artificial Sequence
  Synthetic Polynucleotide
tgccctggatgggaa

SEQ ID NO: 74 - DNA, 21 bp, Artificial Sequence
  Synthetic Polynucleotide
tgcttgacacctggatgggaa

SEQ ID NO: 75 - DNA, 15 bp, Artificial Sequence
  Synthetic Polynucleotide
tcctgagcttgaagt

SEQ ID NO: 76 - DNA, 15 bp, Artificial Sequence
  Synthetic Polynucleotide
tcctgagcttgaagt

SEQ ID NO: 77 - DNA, 15 bp, Artificial Sequence
  Synthetic Polynucleotide
ttctggcggggaagt

SEQ ID NO: 78 - DNA, 21 bp, Artificial Sequence
  Synthetic Polynucleotide
ctcctattgggggtttcctat

SEQ ID NO: 79 - DNA, 26 bp, Artificial Sequence
  Synthetic Polynucleotide
accccctctaccccctctacccctct

SEQ ID NO: 80 - DNA, 12 bp, Artificial Sequence
  Synthetic Polynucleotide
cctggatgggaa

SEQ ID NO: 81 - DNA, 15 bp, Artificial Sequence
  Synthetic Polynucleotide
ttctggcggggaagt

SEQ ID NO: 82 - DNA, 21 bp, Artificial Sequence
  Synthetic Polynucleotide
ctcctattgggggtttcctat

SEQ ID NO: 83 - DNA, 26 bp, Artificial Sequence
  Synthetic Polynucleotide
accccctctaccccctctacccctct

SEQ ID NO: 84 - DNA, 12 bp, Artificial Sequence
  Synthetic Polynucleotide
cctggatgggaa

SEQ ID NO: 85 - DNA, 12 bp, Artificial Sequence
  Synthetic Polynucleotide
cctggatgggaa

SEQ ID NO: 86 - DNA, 12 bp, Artificial Sequence
  Synthetic Polynucleotide
cctggatgggaa

SEQ ID NO: 87 - DNA, 12 bp, Artificial Sequence
  Synthetic Polynucleotide
cctggatgggaa

SEQ ID NO: 88 - DNA, 12 bp, Artificial Sequence
  Synthetic Polynucleotide
cctggatgggaa

SEQ ID NO: 89 - DNA, 12 bp, Artificial Sequence
  Synthetic Polynucleotide
cctggatgggaa

SEQ ID NO: 90 - DNA, 12 bp, Artificial Sequence
  Synthetic Polynucleotide
cctggatgggaa

SEQ ID NO: 91 - DNA, 15 bp, Artificial Sequence
  Synthetic Polynucleotide
ttctggcggggaagt

SEQ ID NO: 92 - DNA, 21 bp, Artificial Sequence
  Synthetic Polynucleotide
ctcctattgggggtttcctat

SEQ ID NO: 93 - DNA, 26 bp, Artificial Sequence
  Synthetic Polynucleotide
accccctctaccccctctacccctct

SEQ ID NO: 94 - DNA, 12 bp, Artificial Sequence
  Synthetic Polynucleotide
cctggatgggaa

SEQ ID NO: 95 - DNA, 15 bp, Artificial Sequence
  Synthetic Polynucleotide
ttctggcggggaagt

SEQ ID NO: 96 - DNA, 21 bp, Artificial Sequence
  Synthetic Polynucleotide
ctcctattgggggtttcctat

SEQ ID NO: 97 - DNA, 26 bp, Artificial Sequence
  Synthetic Polynucleotide
accccctctaccccctctacccctct

SEQ ID NO: 98 - DNA, 12 bp, Artificial Sequence
  Synthetic Polynucleotide
cctggatgggaa

SEQ ID NO: 99 - DNA, 15 bp, Artificial Sequence
  Synthetic Polynucleotide
tcctgagcttgaagt

SEQ ID NO: 100 - DNA, 15 bp, Artificial Sequence
  Synthetic Polynucleotide
tcctgagcttgaagt

SEQ ID NO: 101 - DNA, 20 bp, Artificial Sequence
  Synthetic Polynucleotide
tccatgacgttcctgacgtt

SEQ ID NO: 102 - DNA, 20 bp, Artificial Sequence
  Synthetic Polynucleotide
tccatgacgttcctgatgct

SEQ ID NO: 103 - DNA, 12 bp, Artificial Sequence
  Synthetic Polynucleotide
cctggatgggaa

SEQ ID NO: 104 - DNA, 12 bp, Artificial Sequence
  Synthetic Polynucleotide
cctggatgggaa

SEQ ID NO: 105 - DNA, 12 bp, Artificial Sequence
  Synthetic Polynucleotide
cctggatgggaa

SEQ ID NO: 106 - DNA, 12 bp, Artificial Sequence
  Synthetic Polynucleotide
cctggatgggaa

SEQ ID NO: 107 - DNA, 12 bp, Artificial Sequence
  Synthetic Polynucleotide
cctggatgggaa

SEQ ID NO: 108 - DNA, 12 bp, Artificial Sequence
  Synthetic Polynucleotide
cctggatgggaa

SEQ ID NO: 109 - DNA, 15 bp, Artificial Sequence
  Synthetic Polynucleotide
tcctggaggggttgt

SEQ ID NO: 110 - DNA, 18 bp, Artificial Sequence
  Synthetic Polynucleotide
tgctcctggaggggttgt

SEQ ID NO: 111 - DNA, 13 bp, Artificial Sequence
  Synthetic Polynucleotide
tgctggatgggaa

SEQ ID NO: 112 - DNA, 15 bp, Artificial Sequence
  Synthetic Polynucleotide
tgccctggatgggaa

SEQ ID NO: 113 - DNA, 21 bp, Artificial Sequence
  Synthetic Polynucleotide
tgcttgacacctggatgggaa

SEQ ID NO: 114 - DNA, 13 bp, Artificial Sequence
  Synthetic Polynucleotide
tgctggatgggaa

SEQ ID NO: 115 - DNA, 15 bp, Artificial Sequence
  Synthetic Polynucleotide
tgccctggatgggaa

SEQ ID NO: 116 - DNA, 21 bp, Artificial Sequence
  Synthetic Polynucleotide
tgcttgacacctggatgggaa

SEQ ID NO: 117 - DNA, 15 bp, Artificial Sequence
  Synthetic Polynucleotide
tcctgagcttgaagt

SEQ ID NO: 118 - DNA, 15 bp, Artificial Sequence
  Synthetic Polynucleotide
tcctgagcttgaagt

SEQ ID NO: 119 - DNA, 20 bp, Artificial Sequence
  Synthetic Polynucleotide
tccatgacgttcctgacgtt

SEQ ID NO: 120 - DNA, 20 bp, Artificial Sequence
  Synthetic Polynucleotide
tccatgacgttcctgatgct